SEQUENCE LISTING (36 sequences)

SEQ ID NO: 1 - protein, 118 aa, synthetic construct
  REGION 1..118: note = Synthetic Construct
EVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSS

SEQ ID NO: 2 - protein, 113 aa, synthetic construct
  REGION 1..113: note = Synthetic Construct
DIVMTQSPDSLAVSLGERATINCKSSQSVLYAGNNRNYLAWYQQKPGQPPKLLINQASTRASGVPDRFSGSGSGTEFTLIISSLQAEDVAIYYCQQYYTPPLAFGGGTKLEIK

SEQ ID NO: 3 - protein, 445 aa, synthetic construct
  REGION 1..445: note = Synthetic Construct
EVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 220 aa, synthetic construct
  REGION 1..220: note = Synthetic Construct
DIVMTQSPDSLAVSLGERATINCKSSQSVLYAGNNRNYLAWYQQKPGQPPKLLINQASTRASGVPDRFSGSGSGTEFTLIISSLQAEDVAIYYCQQYYTPPLAFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 5 aa, synthetic construct
  REGION 1..5: note = Synthetic Construct
RAWMN

SEQ ID NO: 6 - protein, 19 aa, synthetic construct
  REGION 1..19: note = Synthetic Construct
RIKRKTDGETTDYAAPVKG

SEQ ID NO: 7 - protein, 7 aa, synthetic construct
  REGION 1..7: note = Synthetic Construct
SNRAFDI

SEQ ID NO: 8 - protein, 17 aa, synthetic construct
  REGION 1..17: note = Synthetic Construct
KSSQSVLYAGNNRNYLA

SEQ ID NO: 9 - protein, 7 aa, synthetic construct
  REGION 1..7: note = Synthetic Construct
QASTRAS

SEQ ID NO: 10 - protein, 9 aa, synthetic construct
  REGION 1..9: note = Synthetic Construct
QQYYTPPLA

SEQ ID NO: 11 - protein, 7 aa, synthetic construct
  REGION 1..7: note = Synthetic Construct
GLTFERA

SEQ ID NO: 12 - protein, 8 aa, synthetic construct
  REGION 1..8: note = Synthetic Construct
KRKTDGET

SEQ ID NO: 13 - protein, 7 aa, synthetic construct
  REGION 1..7: note = Synthetic Construct
SNRAFDI

SEQ ID NO: 14 - protein, 17 aa, synthetic construct
  REGION 1..17: note = Synthetic Construct
KSSQSVLYAGNNRNYLA

SEQ ID NO: 15 - protein, 7 aa, synthetic construct
  REGION 1..7: note = Synthetic Construct
QASTRAS

SEQ ID NO: 16 - protein, 9 aa, synthetic construct
  REGION 1..9: note = Synthetic Construct
QQYYTPPLA

SEQ ID NO: 17 - protein, 8 aa, synthetic construct
  REGION 1..8: note = Synthetic Construct
GLTFERAW

SEQ ID NO: 18 - protein, 10 aa, synthetic construct
  REGION 1..10: note = Synthetic Construct
IKRKTDGETT

SEQ ID NO: 19 - protein, 9 aa, synthetic construct
  REGION 1..9: note = Synthetic Construct
AGSNRAFDI

SEQ ID NO: 20 - protein, 12 aa, synthetic construct
  REGION 1..12: note = Synthetic Construct
QSVLYAGNNRNY

SEQ ID NO: 21 - skipped

SEQ ID NO: 22 - protein, 9 aa, synthetic construct
  REGION 1..9: note = Synthetic Construct
QQYYTPPLA

SEQ ID NO: 23 - protein, 10 aa, synthetic construct
  REGION 1..10: note = Synthetic Construct
GLTFERAWMN

SEQ ID NO: 24 - protein, 12 aa, synthetic construct
  REGION 1..12: note = Synthetic Construct
RIKRKTDGETTD

SEQ ID NO: 25 - protein, 7 aa, synthetic construct
  REGION 1..7: note = Synthetic Construct
SNRAFDI

SEQ ID NO: 26 - protein, 17 aa, synthetic construct
  REGION 1..17: note = Synthetic Construct
KSSQSVLYAGNNRNYLA

SEQ ID NO: 27 - protein, 7 aa, synthetic construct
  REGION 1..7: note = Synthetic Construct
QASTRAS

SEQ ID NO: 28 - protein, 9 aa, synthetic construct
  REGION 1..9: note = Synthetic Construct
QQYYTPPLA

SEQ ID NO: 29 - protein, 6 aa, synthetic construct
  REGION 1..6: note = Synthetic Construct
ERAWMN

SEQ ID NO: 30 - protein, 15 aa, synthetic construct
  REGION 1..15: note = Synthetic Construct
WVGRIKRKTDGETTD

SEQ ID NO: 31 - protein, 8 aa, synthetic construct
  REGION 1..8: note = Synthetic Construct
AGSNRAFD

SEQ ID NO: 32 - protein, 13 aa, synthetic construct
  REGION 1..13: note = Synthetic Construct
LYAGNNRNYLAWY

SEQ ID NO: 33 - protein, 10 aa, synthetic construct
  REGION 1..10: note = Synthetic Construct
LLINQASTRA

SEQ ID NO: 34 - protein, 8 aa, synthetic construct
  REGION 1..8: note = Synthetic Construct
QQYYTPPL

SEQ ID NO: 35 - protein, 444 aa, synthetic construct
  REGION 1..444: note = Synthetic Construct
EVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 36 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

(19) United States
(12) Patent Application Publication
(10) Pub. No.: US 2025/0163150 A1
(43) Pub. Date: May 22, 2025

(54) COMBINATION THERAPIES COMPRISING A KRAS INHIBITOR FOR THE TREATMENT OF CANCER

(71) Applicant: I-Mab Biopharma US Limited, Rockville, MD (US)

(72) Inventors: Yanni ZHANG, Shanghai (CN); Ke XU, Shanghai (CN); Zhengyi WANG, Shanghai (CN)

(73) Assignee: I-Mab Biopharma US Limited, Rockville, MD (US)

(21) Appl. No.: 18/842,767

(22) PCT Filed: Mar. 3, 2023
(86) PCT No.: PCT/CN2023/079640

§ 371 Date: Aug. 29, 2024

(30) Foreign Application Priority Data
Mar. 4, 2022 (WO) PCT/CN2022/079307

(51) Int. Cl. C07K 16/28; A61K 31/519; A61P 35/00

(52) U.S. Cl. CPC C07K 16/2803; A61K 31/519; A61P 35/00

(57) ABSTRACT

Provided herein are methods of treating a KRAS mutant cancer (e.g., a KRAS G12C mutant cancer or a KRAS G12D mutant cancer) in a subject, comprising administering to the subject an effective amount of an agent that blocks the interaction between CD47 and SIRPα and an effective amount of a KRAS inhibitor (e.g., a KRAS G12C inhibitor wherein the cancer is a KRAS G12C mutant cancer, or a KRAS G12D inhibitor wherein the cancer is a KRAS G12D mutant cancer). Also provided are methods of stimulating phagocytosis of a population of cancer cells that express a KRAS mutant protein (e.g., a KRAS G12C mutant protein or a KRAS G12D mutant protein) by macrophages, comprising contacting the population with an effective amount of a therapeutic agent that blocks the interaction between CD47 and SIRPα and an effective amount of a KRAS inhibitor (e.g., a KRAS G12C inhibitor wherein the KRAS mutant protein is a KRAS G12C mutant protein, or a KRAS G12D inhibitor wherein the KRAS mutant protein is a KRAS G12D mutant protein).

### CROSS REFERENCE TO RELATED APPLICATIONS

This application claims the priority benefit of International Application No. PCT/CN2022/079307, filed Mar. 4, 2022, the contents of which are incorporated herein by reference in their entirety.

### REFERENCE TO AN ELECTRONIC SEQUENCE LISTING

The contents of the electronic sequence listing (233002000741SEQLIST.xml; Size: 45,965 bytes; and Date of Creation: Feb. 15, 2023) is herein incorporated by reference in its entirety.

### FIELD OF THE INVENTION

The present application relates to combination therapies for the treatment of KRAS mutant cancers, e.g., non-small cell lung cancer (NSCLC), colon cancer, colorectal cancer, and others.

### BACKGROUND OF THE INVENTION

KRAS mutations play a role in some of the most common and deadly cancers, including lung, colon, colorectal, and rectal cancers. G12C is a single point mutation with a glycine-to-cysteine substitution at codon 12 of the KRAS protein. This substitution favors the active, GTP-bound conformation of KRAS, amplifying signaling pathways that lead to oncogenesis. KRAS G12C is particularly prevalent in non-small cell lung cancer (NSCLC), which makes up about 85% of all lung cancer cases in the U.S. Approximately 13% of Americans with NSCLC have the KRAS G12C mutation, and there are about 23,000 new cases of KRAS G12C NSCLC diagnosed every year in the U.S. alone. Clinical trials of KRAS G12C allele-specific inhibitors adagrasib and sotorasib have shown promising activity in cancers expressing the KRAS G12C mutant protein. However, the clinical trial data also indicates that there is significant variation in response among patients treated with KRAS G12C inhibitors and that KRAS G12C inhibitor monotherapy is unlikely to be sufficient to elicit a sustained therapeutic response. G12D is another single point mutation with a glycine-to-aspartic acid substitution at codon 12 of the KRAS protein. The G12D mutation accounts for about 41% of all the G12 mutations and is the most prevalent G12 mutation in colorectal cancer and pancreatic ductal adenocarcinoma (PDAC). Accordingly, there is a continuing need in the art for methods of treating cancers expressing the KRAS G12C mutant protein or the KRAS G12D mutant protein.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY OF THE INVENTION

Provided herein is a method of treating cancer in a subject, comprising administering to the subject an effective amount of an agent that blocks the interaction between CD47 and SIRPα and an effective amount of a KRAS inhibitor, wherein the cancer comprises one or more cancer cells that express a KRAS mutant protein. Also provided herein is a method of stimulating phagocytosis of a population of cancer cells by macrophages, comprising contacting the population with an effective amount of an agent that blocks the interaction between CD47 and SIRPα and an effective amount of a KRAS inhibitor, wherein the population of cancer cells comprises one or more cancer cells that express a KRAS mutant protein.

In some embodiments, the agent that blocks the interaction between CD47 and SIRPα is a polypeptide that binds CD47. In some embodiments, the polypeptide that binds CD47 is anti-CD47 antibody or immunologically active fragment thereof. In some embodiments, the anti-CD47 antibody or immunologically active fragment thereof is CC-90002, 5F9, LQ001, HLX24, TI-061, AO-176, SRF-231, IBI-188, IMC-002, SHR-1603, STI-6643, ZL-1201, or an immunologically active fragment of any one of the preceding.

In some embodiments, the anti-CD47 antibody or immunologically active fragment thereof comprises three complementarity determining regions (CDRs) of a heavy chain variable domain (V_{H}) set forth in SEQ ID NO: 1 and three CDRs of a light chain variable domain (V_{L}) set forth in SEQ ID NO: 2. In some embodiments, the anti-CD47 antibody or immunologically active fragment thereof comprises (a) a V_{H} that comprises (1) a CDR-H1 comprising RAWMN (SEQ ID NO: 5); (2) a CDR-H2 comprising RIKRKTDGETTDYAAPVKG (SEQ ID NO: 6); and (3) a CDR-H3 comprising SNRAFDI (SEQ ID NO: 7) and (b) a V_{L} that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10), wherein the CDR sequences are defined according to the Kabat numbering system. In some embodiments, the V_{H} domain of the anti-CD47 antibody or immunologically active fragment thereof comprises an amino acid sequence that has at least 95% identity to SEQ ID NO: 1, and the V_{L} of the anti-CD47 antibody or immunologically active fragment thereof comprises an amino acid sequence that has at least 95% identity to SEQ ID NO: 2. In some embodiments, the N-terminal amino acid of the V_{H} domain is E and the C-terminal amino acid of the V_{H} domain is S. In some embodiments, (a) the N-terminal amino acid of the V_{H} domain corresponds to position H1 and the C-terminal amino acid of the V_{H} domain corresponds to position H113, according to the Kabat numbering system; (b) the N-terminal amino acid of the V_{H} domain corresponds to position H1 and the C-terminal amino acid of the V_{H} domain corresponds to position H113, according to the Chothia numbering system; or (c) the N-terminal amino acid of the V_{H} domain corresponds to position H1 and the C-terminal amino acid of the V_{H} domain corresponds to position H128, according to the IMGT numbering system. In some embodiments, the N-terminal amino acid of the V_{H} domain corresponds to amino acid 1 of SEQ ID NO: 1, and the C-terminal amino acid of the V_{H} domain corresponds to amino acid 118 of SEQ ID NO: 1. In some embodiments, the anti-CD47 antibody is a full-length antibody. In some embodiments, the full length anti-CD47 antibody comprises a human IgG4 Fc region or a variant thereof that comprises an S228P substitution, wherein the amino acid numbering is according to the EU index. In some embodiments, the full length anti-CD47 antibody comprises a heavy chain that comprises SEQ ID NO: 3 or SEQ ID NO: 35 and a light chain that comprises SEQ ID NO: 4.

In some embodiments, the KRAS inhibitor is a KRAS G12C inhibitor, and the KRAS mutant protein is a KRAS G12C mutant protein. In some embodiments, the KRAS G12C inhibitor is an antibody, a peptide, a protein, an antisense oligonucleotide, or a small molecule that inhibits the activity of the KRAS G12C mutant protein. In some embodiments, the KRAS G12C inhibitor is a small molecule. In some embodiments, the small molecule is selected from the group consisting of: AMG 510, MRTX849, JAB-21822, GDC-6036, JDQ443, D-1553, GH35, GFH925, BPI-421286, and LY3537982. In some embodiments, the small molecule is AMG 510 or MRTX849.

In some embodiments, the KRAS inhibitor is a KRAS G12D inhibitor, and the KRAS mutant protein is a KRAS G12D mutant protein. In some embodiments, the KRAS G12D inhibitor is an antibody, a peptide, a protein, an antisense oligonucleotide, or a small molecule that inhibits the activity of the KRAS G12D mutant protein. In some embodiments, the KRAS G12D inhibitor is a small molecule. In some embodiments, the small molecule is MTRX1122.

In some embodiments of any of the methods herein, the cancer is lung cancer, colon cancer, colorectal cancer, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal cancer. In some embodiments, the cancer is a KRAS G12C cancer. In some embodiments, the KRAS G12C caner is lung cancer. In some embodiments, the lung cancer is lung adenocarcinoma or non-small cell lung cancer (NSCLC). In some embodiments, the lung cancer is non-small cell lung cancer (NSCLC). In some embodiments, the cancer is a KRAS G12D cancer. In some embodiments, the KRAS G12D cancer is colorectal cancer or colon cancer.

In some embodiments of any of the methods herein, the anti-CD47 antibody or immunologically active fragment thereof and the KRAS inhibitor are administered simultaneously. In some embodiments, the anti-CD47 antibody or immunologically active fragment thereof and the KRAS inhibitor are administered sequentially. In some embodiments, the anti-CD47 antibody or immunologically active fragment thereof is administered prior to the KRAS inhibitor. In some embodiments, the KRAS inhibitor is administered prior to the anti-CD47 antibody or immunologically active fragment thereof.

In some embodiments, the subject is human.

In some embodiments of the methods herein, the one or more cancer cells that express a KRAS mutant protein are lung cancer cells, colon cancer cells, colorectal cancer cells, pancreatic cancer cells, cholangiocarcinoma cells, endometrial cancer cells, ovarian cancer cells, peritoneal cancer cells, bladder cancer cells, gastric cancer cells, thyroid cancer cells, melanoma cells, breast cancer cells, head and neck cancer cells, multiple myeloma cells, acute myeloid leukemia (AML) cells, uterine cancer cells, gastro-esophageal cancer cells, or rectal cancer cells. In some embodiments, the KRAS inhibitor is a KRAS G12C inhibitor, the one or more cancer cells that express a KRAS mutant protein express a KRAS G12C mutant protein, the one or more cancer cells that express the KRAS G12C mutant protein are lung cancer cells, and the is lung cancer cells are lung adenocarcinoma cells or non-small cell lung cancer (NSCLC) cells. In some embodiments, the lung cancer cells are NSCLC cells. In some embodiments, the KRAS inhibitor is a KRAS G12D inhibitor, the one or more cancer cells that express a KRAS mutant protein express a KRAS G12D mutant protein, and the one or more cancer cells that express the KRAS G12D mutant protein are colon cancer cells or colorectal cancer cells.

In some embodiments, provided is a kit for treating cancer in a human subject comprising: (a) an agent that blocks the interaction between CD47 and SIRPα, and (b) instructions for administering an effective amount of the agent and an effective amount of a KRAS inhibitor to a subject who has a cancer that comprises one or more cancer cells that express a KRAS mutant protein. In some embodiments, the polypeptide is polypeptide that binds CD47. In some embodiments, the polypeptide that binds CD47 is anti-CD47 antibody or immunologically active fragment thereof. In some embodiments, the anti-CD47 antibody or immunologically active fragment thereof comprises (a) a V_{H} that comprises (1) a CDR-H1 comprising RAWMN (SEQ ID NO: 5); (2) a CDR-H2 comprising RIKRKTDGETTDYAAPVKG (SEQ ID NO: 6); and (3) a CDR-H3 comprising SNRAFDI (SEQ ID NO: 7) and (b) a V_{L} that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10), wherein the CDR sequences are defined according to the Kabat numbering system. In some embodiments, the V_{H} domain of the anti-CD47 antibody or immunologically active fragment thereof comprises an amino acid sequence that has at least 95% identity to SEQ ID NO: 1, and the V_{L} of the anti-CD47 antibody or immunologically active fragment thereof comprises an amino acid sequence that has at least 95% identity to SEQ ID NO: 2. In some embodiments, the N-terminal amino acid of the V_{H} domain is E and the C-terminal amino acid of the V_{H} domain is S. In some embodiments, the anti-CD47 antibody is a full-length antibody. In some embodiments, the full length anti-CD47 antibody comprises a heavy chain that comprises SEQ ID NO: 3 or SEQ ID NO: 35 and a light chain that comprises SEQ ID NO: 4. In some embodiments, the kit further comprises a KRAS inhibitor. In some embodiments, the KRAS inhibitor is a KRAS G12C inhibitor and the cancer comprises one or more cancer cells that express a KRAS G12C mutant protein. In some embodiments, the KRAS G12C inhibitor is an antibody, a peptide, a protein, an antisense oligonucleotide, or a small molecule that inhibits the activity of the KRAS G12C mutant protein. In some embodiments, the KRAS G12C inhibitor is a small molecule inhibitor. In some embodiments, the small molecule is selected from the group consisting of: AMG 510, MRTX849, JAB-21822, GDC-6036, JDQ443, D-1553, GH35, GFH925, BPI-421286, and LY3537982. In some embodiments, the small molecule is AMG 510 or MRTX849. In some embodiments, the KRAS inhibitor is a KRAS G12D inhibitor and the cancer comprises one or more cancer cells that express a KRAS G12D mutant protein. In some embodiments, the KRAS G12D inhibitor is an antibody, a peptide, a protein, an antisense oligonucleotide, or a small molecule that inhibits the activity of the KRAS G12D mutant protein. In some embodiments, the KRAS G12D inhibitor is a small molecule inhibitor. In some embodiments, the small molecule MRTX1133. In some embodiments, the cancer that comprises one or more cancer cells that express a KRAS mutant protein is lung cancer, colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal adenocarcinoma.

In some embodiments, provided is pharmaceutical composition comprising an agent that blocks the interaction between CD47 and SIRPα and a KRAS inhibitor. In some embodiments, the agent that blocks the interaction between CD47 and SIRPα is an anti-CD47 antibody or immunologically active fragment thereof.

In some embodiments, the anti-CD47 antibody or immunologically active fragment thereof is CC-90002, 5F9, LQ001, HLX24, TI-061, AO-176, SRF-231, IBI-188, IMC-002, SHR-1603, STI-6643, ZL-1201, or an immunologically active fragment. In some embodiments, the anti-CD47 antibody or immunologically active fragment thereof comprises (a) a V_{H} that comprises (1) a CDR-H1 comprising RAWMN (SEQ ID NO: 5); (2) a CDR-H2 comprising RIKRKTDGETTDYAAPVKG (SEQ ID NO: 6); and (3) a CDR-H3 comprising SNRAFDI (SEQ ID NO: 7) and (b) a V_{L} that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10), wherein the CDR sequences are defined according to the Kabat numbering system. In some embodiments, the KRAS inhibitor is a KRAS G12C inhibitor. In some embodiments, the KRAS G12C inhibitor is selected from the group consisting of AMG 510, MRTX849, JAB-21822, GDC-6036, JDQ443, D-1553, GH35, GFH925, BPI-421286, and LY3537982. In some embodiments, the KRAS inhibitor is a KRAS G12D inhibitor. In some embodiments, the KRAS G12D inhibitor is MTRX1133.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTIONS OF THE DRAWINGS

FIG. 1 provides the results of experiments that were performed to compare the efficacy of (a) PBS (control), (b) AMG-510 monotherapy, (c) TJC4 monotherapy, and (d) AMG-510 in combination with TJC4 in inhibiting tumor growth in mice bearing human NCI-H358 non-small cell lung cancer tumor xenografts. The NCI-H358 cell line has the KRAS G12C mutation.

FIG. 2 provides of experiments that were performed to compare the weight of tumors from mice bearing human NCI-H358 non-small cell lung cancer tumor xenografts following treatment with (a) PBS (control), (b) AMG-510 monotherapy, (c) TJC4 monotherapy, or (d) AMG-510 in combination with TJC4.

FIG. 3A provides the results of flow cytometry experiments that were performed to determine the levels of calreticulin (“CALR”) in tumors from mice bearing human NCI-H358 non-small cell lung cancer tumor xenografts following treatment with (a) PBS (control), (b) AMG-510 monotherapy, (c) TJC4 monotherapy, or (d) AMG-510 in combination with TJC4.

FIG. 3B provides the results of flow cytometry experiments that were performed to determine the ratio of M1 macrophages:M2 macrophages in tumors from mice bearing human NCI-H358 non-small cell lung cancer tumor xenografts following treatment with (a) PBS (control), (b) AMG-510 monotherapy, (c) TJC4 monotherapy, or (d) AMG-510 in combination with TJC4.

FIG. 3C provides the results of flow cytometry experiments that were performed to determine the percentage of M1 macrophages in tumors from mice bearing human NCI-H358 non-small cell lung cancer tumor xenografts following treatment with (a) PBS (control), (b) AMG-510 monotherapy, (c) TJC4 monotherapy, or (d) AMG-510 in combination with TJC4.

FIG. 3D provides the results of multiplex immunohistochemistry experiments that were performed to determine the percentages of total macrophages, M1 macrophages, and M2 macrophages in tumors from mice bearing human NCI-H358 non-small cell lung cancer tumor xenografts following treatment with (a) PBS (control), (b) AMG-510 monotherapy, (c) TJC4 monotherapy, or (d) AMG-510 in combination with TJC4.

FIG. 3E provides the results of multiplex immunohistochemistry experiments that were performed to determine the ratio of M1 macrophages:M2 macrophages, as determined via multiplexed immunohistochemistry, in tumors from mice bearing human NCI-H358 non-small cell lung cancer tumor xenografts following treatment with (a) PBS (control), (b) AMG-510 monotherapy, (c) TJC4 monotherapy, or (d) AMG-510 in combination with TJC4.

FIG. 3F provides multiplex immunofluorescence images of tumors from mice bearing human NCI-H358 non-small cell lung cancer tumor xenografts following treatment with (a) PBS (control), (b) AMG-510 monotherapy, (c) TJC4 monotherapy, or (d) AMG-510 in combination with TJC4.

FIG. 4A provides the results of in vitro phagocytosis assays that were performed using NCI-H358 cells that were incubated with M0 macrophage, 1 μg/ml TJC4, and 0.1 μm AMG 150.

FIG. 4B provides the results of in vitro phagocytosis assays that were performed using NCI-H358 cells that were incubated with M0 macrophage, 1 μg/ml TJC4, and 1 μm AMG 150.

FIG. 4C provides the results of in vitro phagocytosis assays that were performed using NCI-H358 cells that were incubated with M0 macrophage, 1 μg/ml TJC4, and 10 μm AMG 150.

FIG. 5A provides the results of flow cytometry experiments that were performed to assess the effects of DMSO, MRTX1133, TJC4, and MRTX1133+TJC4 on the phagocytosis of human LS1780 colon cancer cells by macrophages in vitro.

FIG. 5B provides quantitative results of the data shown in FIG. 5A.

FIG. 6A provides the results of flow cytometry experiments that were performed to assess the effects of DMSO, MRTX1133, TJC4, and MRTX1133+TJC4 on apoptosis of human LS1780 colon cancer cells in vitro.

FIG. 6B provides quantitative results of the data shown in FIG. 6A.

FIG. 7A provides a schematic of the experimental procedure described in Example 4.

FIG. 7B provides the results of experiments that were performed to assess the anti-tumor efficacy of PBS, MRTX1133, TJC4, and MRTX1133+TJC4 in mice bearing human LS180 colon cancer tumor xenografts.

FIG. 7C provides luciferase images of human LS180 colon cancer tumor xenografts in mice treated with PBS, MRTX1133, TJC4, or MRTX1133+TJC4.

### DETAILED DESCRIPTION OF THE INVENTION

### Definitions

Before describing the embodiments in detail, it is to be understood that the present disclosure is not limited to particular compositions or biological systems, which can, of course, vary. It is also to be understood that the terminology used herein is for the purpose of describing particular embodiments only and is not intended to be limiting.

As used in this specification and the appended claims, the singular forms “a,” “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure include “comprising,” “consisting,” and “consisting essentially of” aspects and embodiments.

The term “CD47” (which is also known as Integrin Associated Protein (IAP), Antigenic Surface Determinant Protein OA3, OA3, CD47 Antigen, Rh-Related Antigen, Integrin-Associated Signal Transducer, Antigen Identified By Monoclonal Antibody 1D8, CD47 glycoprotein) preferably refers to human CD47 and, in particular, to a protein comprising the amino acid sequence

(SEQ ID NO: 36)
MWPLVAALLL GSACCGSAQL LFNKTKSVEF TFCNDTVVIP
CFVTNMEAQN TTEVYVKWKF KGRDIYTFDG ALNKSTVPTD
FSSAKIEVSQ LLKGDASLKM DKSDAVSHTG NYTCEVTELT
REGETIIELK YRVVSWFSPN ENILIVIFPI FAILLFWGQF
GIKTLKYRSG GMDEKTIALL VAGLVITVIV IVGAILFVPG
EYSLKNATGL GLIVISTGIL ILLHYYVFST AIGLTSFVIA
ILVIQVIAYI LAVVGLSLCI AACIPMHGPL LISGLSILAL
AQLLGLVYMK FVASNQKTIQ PPRKAVEEPL NAFKESKGMM
NDE

or a variant of said amino acid sequence. The term “CD47” also refers to any post translationally modified variants and conformation variants.

As used herein, the term “antibody” is used in the broadest sense and specifically covers intact antibodies (e.g., full length antibodies), antibody fragments (including without limitation Fab, F(ab′)2, scFv, scFv-Fc, single domain antibodies), monoclonal antibodies, and polyclonal antibodies, so long as they exhibit the desired biological activity (e.g., epitope binding).

As used herein, the term “isolated” antibody may refer to an antibody that is substantially free of other cellular material. In one embodiment, an isolated antibody is substantially free of other proteins from the same species. In another embodiment, an isolated antibody is expressed by a cell from a different species and is substantially free of other proteins from the different species. In some embodiments, an “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. An antibody may be rendered substantially free of naturally associated components (or components associated with the cellular expression system used to produce the antibody) by isolation, using protein purification techniques well known in the art. In some embodiments, the antibody will be purified (1) to greater than 75% by weight of antibody as determined by the Lowry method, and most preferably more than 80%, 90%, 95% or 99% by weight, or (2) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the term “epitope” means any antigenic determinant on an antigen to which the paratope of an antibody binds. Epitopic determinants usually consist of chemically active surface groupings of molecules such as amino acids or sugar side chains and usually have specific three-dimensional structural characteristics, as well as specific charge characteristics.

As used herein, the term “native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond (also termed a “VH/VL pair”), while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains. See, e.g., Chothia et al., J. Mol. Biol., 186:651 (1985); Novotny and Haber, Proc. Natl. Acad. Sci. U.S.A., 82:4592 (1985).

As used herein, the term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies. See, e.g., Kabat et al., Sequences of Proteins of Immunological Interest, Fifth Edition, National Institute of Health, Bethesda, Md. (1991). The constant domains are not involved directly in binding an antibody to an antigen, but exhibit various effector functions, such as participation of the antibody in antibody-dependent cellular toxicity. Variable region sequences of interest include the humanized variable region sequences for CD47 antibodies described in detail elsewhere herein.

The term “hypervariable region (HVR)” or “complementarity determining region (CDR)” may refer to the subregions of the VH and VL domains characterized by enhanced sequence variability and/or formation of defined loops. These include three CDRs in the VH domain (H1, H2, and H3) and three CDRs in the VL domain (L1, L2, and L3). H3 is believed to be critical in imparting fine binding specificity, with L3 and H3 showing the highest level of diversity. See Johnson and Wu, in Methods in Molecular Biology 248:1-25 (Lo, ed., Human Press, Totowa, N.J., 2003).

A number of CDR/HVR delineations are known. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops and are used by Oxford Molecular's AbM antibody modeling software. The “contact” HVRs are based on an analysis of the available complex crystal structures. The residues from each of these HVRs/CDRs are noted below. “Framework” or “FR” residues are those variable domain residues other than the HVR/CDR residues.

| Loop | Kabat | AbM | Chothia | Contact | |
|---|---|---|---|---|---|
| L1 | L24-L34 | L24-L34 | L26-L32 | L30-L36 | |
| L2 | L50-L56 | L50-L56 | L50-L52 | L46-L55 | |
| L3 | L89-L97 | L89-L97 | L91-L96 | L89-L96 | |
| H1 | H31-H35B | H26-H35B | H26-H32 | H30-H35B | (Kabat Numbering) |
| H1 | H31-H35 | H26-H35 | H26-H32 | H30-H35 | (Chothia Numbering) |
| H2 | H50-H65 | H50-H58 | H53-H55 | H47-H58 | |
| H3 | H95-H102 | H95-H102 | H96-H101 | H93-H101 | |

“Extended” HVRs are also known: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH (Kabat numbering).

“Numbering according to Kabat” may refer to the numbering system used for heavy chain variable domains or light chain variable domains of the compilation of antibodies in Kabat et al., supra. The actual linear amino acid sequence may contain fewer or additional amino acids corresponding to a shortening of, or insertion into, a FR or HVR of the variable domain. The Kabat numbering of residues may be determined for a given antibody by alignment at regions of homology of the sequence of the antibody with a “standard” Kabat numbered sequence. Typically, the Kabat numbering is used when referring to a residue in the variable domains (approximately residues 1-107 of the light chain and residues 1-113 of the heavy chain), whereas the EU numbering system or index (e.g., the EU index as in Kabat, numbering according to EU IgG1) is generally used when referring to a residue in the heavy chain constant region.

As used herein, a “monoclonal” antibody refers to an antibody obtained from a population of substantially homogeneous antibodies, e.g., substantially identical but allowing for minor levels of background mutations and/or modifications. “Monoclonal” denotes the substantially homogeneous character of antibodies and does not require production of the antibody by any particular method. In some embodiments, a monoclonal antibody is selected by its HVR, VH, and/or VL sequences and/or binding properties, e.g., selected from a pool of clones (e.g., recombinant, hybridoma, or phage-derived). A monoclonal antibody may be engineered to include one or more mutations, e.g., to affect binding affinity or other properties of the antibody, create a humanized or chimeric antibody, improve antibody production and/or homogeneity, engineer a multispecific antibody, resultant antibodies of which are still considered to be monoclonal in nature. A population of monoclonal antibodies may be distinguished from polyclonal antibodies as the individual monoclonal antibodies of the population recognize the same antigenic site. A variety of techniques for production of monoclonal antibodies are known; see, e.g., the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

“Chimeric” antibodies may refer to an antibody with one portion of the heavy and/or light chain from a particular isotype, class, or organism and another portion from another isotype, class, or organism. In some embodiments, the variable region will be from one source or organism, and the constant region will be from another.

“Humanized antibodies” may refer to antibodies with predominantly human sequence and a minimal amount of non-human (e.g., mouse or chicken) sequence. In some embodiments, a humanized antibody has one or more HVR sequences (bearing a binding specificity of interest) from an antibody derived from a non-human (e.g., mouse or chicken) organism grafted onto a human recipient antibody framework (FR). In some embodiments, non-human residues are further grafted onto the human framework (not present in either source or recipient antibodies), e.g., to improve antibody properties. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. See Jones et al., Nature 321:522-525 (1986); Riechmann et al., Nature 332:323-329 (1988); and Presta, Curr. Op. Struct. Biol. 2:593-596 (1992).

A “human” antibody may refer to an antibody having an amino acid sequence which corresponds to that of an antibody produced by a human and/or has been made using any of the techniques for making human antibodies as disclosed herein. Human antibodies can be produced using various techniques known in the art, including phage-display libraries. Hoogenboom and Winter, J. Mol. Biol., 227:381 (1991); Marks et al., J. Mol. Biol., 222:581 (1991); preparation of human monoclonal antibodies as described in Cole et al., Monoclonal Antibodies and Cancer Therapy, Alan R. Liss, p. 77 (1985); Boerner et al., J. Immunol., 147(1):86-95 (1991); and by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology) or chickens with human immunoglobulin sequence(s) (see, e.g., WO2012162422, WO2011019844, and WO2013059159).

There are five major classes of immunoglobulins: IgA, IgD, IgE, IgG, and IgM, and several of these can be further divided into subclasses (isotypes), e.g., IgG1, IgG2, IgG3, IgG4, IgA1, IgA2. The heavy-chain constant domains that correspond to the different classes of immunoglobulins are called α, δ, ε, γ, and μ, respectively. The subunit structures and three-dimensional configurations of different classes of immunoglobulins are well known.

As used herein, the term “antibody fragment,” and all grammatical variants thereof, are defined as a portion of an intact antibody comprising the antigen binding site or variable region of the intact antibody which, in certain instances, is free of the constant heavy chain domains (i.e. CH2, CH3, and/or CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules, (2) single chain polypeptides containing only one light chain variable domain, or a fragment thereof that contains the three CDRs of the light chain variable domain, without an associated heavy chain moiety, and (3) single chain polypeptides containing only one heavy chain variable region, or a fragment thereof containing the three CDRs of the heavy chain variable region, without an associated light chain moiety; and multi-specific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. “Fv” is the minimum antibody fragment which contains a complete antigen-recognition and -binding site. In a two-chain Fv species, this region consists of a dimer of one heavy- and one light-chain variable domain in tight, non-covalent association. In a single-chain Fv species (scFv), one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. See, e.g., Pluckthün, in The Pharmacology of Monoclonal Antibodies, Vol. 113, Rosenburg and Moore eds., Springer-Verlag, New York, pp. 269-315 (1994).

The Fab fragment also contains the constant domain of the light chain and the first constant domain (CH₁) of the heavy chain. Fab′ fragments differ from Fab fragments by the addition of a few residues at the carboxy terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

As used herein, the term “monoclonal antibody” (mAb) refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Each mAb is directed against a single determinant on the antigen. In addition to their specificity, the monoclonal antibodies are advantageous in that they can be synthesized by hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made in an immortalized B cell or hybridoma thereof, or may be made by recombinant DNA methods.

The monoclonal antibodies herein include hybrid and recombinant antibodies produced by splicing a variable (including hypervariable) domain of an CD47 antibody with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′)₂, and Fv), so long as they exhibit the desired biological activity.

The monoclonal antibodies herein specifically include chimeric antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

As used herein, the term “treatment” refers to clinical intervention designed to alter the natural course of the individual or cell being treated during the course of clinical pathology. Desirable effects of treatment include decreasing the rate of disease progression, ameliorating, or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals. In some embodiments, “treating” a disease such as cancer refers to delaying progression of the disease, i.e., deferring, hindering, slowing, retarding, stabilizing, and/or postponing development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “effective amount” is at least the minimum amount required to effect a measurable improvement or prevention of a particular disease (e.g., cancer). An effective amount herein may vary according to factors such as the disease state, age, sex, and weight of the patient, and the ability of a therapeutic agent (or combination of therapeutic agents) to elicit a desired response in the individual. An effective amount is also one in which any toxic or detrimental effects of the treatment are outweighed by the therapeutically beneficial effects. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this disclosure, an effective amount of drug, compound, or pharmaceutical composition is an amount sufficient to accomplish therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, the term “subject” for purposes of treatment refers to any animal classified as a mammal, including humans, domestic and farm animals, and zoo, sports, or pet animals, such as dogs, horses, cats, cows, etc. Preferably, the mammal is human.

All references cited herein, including patent applications and publications, are hereby incorporated by reference in their entirety.

### Overview

The present application is based on the unexpected finding that a combination treatment comprising a KRAS inhibitor and an agent that blocks the interaction between CD47 and SIRPα (e.g., an anti-CD47 antibody) is significantly more effective in inhibiting the growth of tumors comprising cells that express the KRAS mutant protein than either drug alone. Without being bound by theory, the synergistic anti-tumor effect of the combination is at least in part due to the fact that such combination stimulates phagocytosis of tumor cells by macrophages to a greater degree than either drug alone. In some embodiments the KRAS inhibitor is a KRAS G12C inhibitor (e.g., AMG 510) and the cancer or population of cancer cells (e.g., tumor) comprises one or more cells that express the KRAS G12C mutant protein. In some embodiments the KRAS inhibitor is a KRAS G12D inhibitor (e.g., AMG MRTX1133) and the cancer or population of cancer cells (e.g., tumor) comprises one or more cells that express the KRAS G12D mutant protein.

### Methods of Treating Cancer and Stimulating the Phagocytosis of Cancer Cells by Macrophages

Provided herein is a method of treating cancer (i.e., a cancer comprising one or more cancer cells that express a KRAS mutant protein (e.g., a KRAS G12C mutant protein or a KRAS G12D mutant protein) in a subject, comprising administering to the subject an effective amount of an agent (e.g., a therapeutic agent) that blocks the interaction between CD47 (e.g., hCD47) and SIRPα (e.g., hSIRPα) and an effective amount of a KRAS inhibitor (e.g., a KRAS G12C inhibitor wherein the KRAS mutant protein is a KRAS G12C mutant protein, or a KRAS G12D inhibitor wherein the KRAS mutant protein is a KRAS G12D mutant protein. In some embodiments, the subject is a human. Also provided is a method of stimulating phagocytosis of a population of cancer cells that comprises one or more cancer cells that express a KRAS mutant protein (e.g., a KRAS G12C mutant protein or a KRAS G12D mutant protein) by macrophages, comprising contacting the population with an effective amount of an agent (e.g., a therapeutic agent) that blocks the interaction between CD47 (e.g., hCD47) and SIRPα (e.g., hSIRPα) and an effective amount of a KRAS inhibitor (e.g., a KRAS G12C inhibitor wherein the KRAS mutant protein is a KRAS G12C mutant protein, or a KRAS G12D inhibitor wherein the KRAS mutant protein is a KRAS G12D mutant protein). A cancer (or a population of cancer cells) that comprises one or more cancer cells that express a KRAS mutant protein is alternatively referred to herein as “a KRAS-associated cancer” or “a KRAS mutant cancer.” In some embodiments, the KRAS mutant cancer is lung cancer, colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma.

In some embodiments, the agent that blocks the interaction between CD47 (e.g., hCD47) and SIRPα (e.g., hSIRPα) is a polypeptide. In some embodiments, the agent that blocks the interaction between CD47 (e.g., hCD47) and SIRPα (e.g., hSIRPα) is a polypeptide that binds CD47 (e.g., hCD47). In some embodiments, the polypeptide is or comprises an anti-CD47 antibody, an immunologically active fragment thereof, or an antibody-based construct (such as a multispecific construct, e.g., a bispecific antibody). Exemplary anti-CD47 antibodies that find use with the methods are described in further detail below.

In some embodiments, the KRAS inhibitor is a KRAS G12C inhibitor, and the cancer (or a population of cancer cells) comprises one or more cancer cells that express a KRAS G12C mutant protein. Such cancer is alternatively referred to herein as “a KRAS G12C-associated cancer” or “a KRAS G12C mutant cancer.” In some embodiments, the KRAS G12C mutant cancer is lung cancer, colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12C mutant cancer is lung adenocarcinoma or non-small cell lung cancer (NSCLC). In some embodiments, the KRAS G12C inhibitor is, e.g., a polypeptide (such as an antibody), a peptide, an antisense oligonucleotide or a small molecule that inhibits the activity of the KRAS G12C mutant protein. In some embodiments, the KRAS G12C inhibitor is a small molecule. Exemplary small molecule KRAS G12C inhibitors that find use with the methods provided herein include, without limitation, e.g., AMG 510 (Amgen/Beigene), MRTX849 (Mirati/Zai Lab), JAB-21822 (Jacobiopharma), GDC-6036 (Genentech), JDQ443 (Novartis), D-1553 (InventisBio and Merck Sharp & Dohme), GH35 (Genhouse Bio), GFH925 (GenFleet Therapeutics), BPI-421286 (Bettapharma), and LY3537982. Additional details regarding these and other exemplary small molecule KRAS G12C inhibitors are provided below.

In some embodiments, the KRAS inhibitor is a KRAS G12D inhibitor, and the cancer (or a population of cancer cells) comprises one or more cancer cells that express a KRAS G12D mutant protein. Such cancer is alternatively referred to herein as “a KRAS G12D-associated cancer” or “a KRAS G12D mutant cancer.” In some embodiments, the KRAS G12D mutant cancer is lung cancer, colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12D mutant cancer is colorectal cancer, such as colorectal adenocarcinoma, or colon cancer, such as colon adenocarcinoma. In some embodiments, the KRAS G12D inhibitor is, e.g., a polypeptide (such as an antibody), a peptide, an antisense oligonucleotide or a small molecule that inhibits the activity of the KRAS G12D mutant protein. In some embodiments, the KRAS G12D inhibitor is a small molecule. Exemplary small molecule KRAS G12D inhibitors that find use with the methods provided herein include, without limitation, e.g., MRTX1133. In some embodiments, the KRAS G12D inhibitor that finds use with the methods provided herein is or comprises an siRNA that targets an mRNA encoding the KRAS G12D mutant protein (also referred to herein as “a KRAS G12D siRNA”). In some embodiments, the KRAS G12D siRNA is loaded into mesenchymal stromal cell-derived exosomes. Such exosomes are also known as “iExosomes” (see, e.g., clinical trial NCT03608631 and Surana et al. (2022) Journal of Clinical Oncology, 40:4_suppl, TPS633-TPS633). Additional details regarding these KRAS G12D inhibitors are provided below.

In some embodiments, the agent that blocks the interaction between CD47 (e.g., hCD47) and SIRPα (e.g., hSIRPα) and the KRAS inhibitor are administered simultaneously. In some embodiments, “simultaneous administration” means that the agent that blocks the interaction between CD47 (e.g., hCD47) and SIRPα (e.g., hSIRPα) and the KRAS inhibitor (e.g., the KRAS G12C inhibitor or the KRAS G12D inhibitor) are administered with a time separation of no more than about 15 minute(s), such as no more than about any of 10, 5, or 1 minutes. In some embodiments, simultaneous administration of the agent that blocks the interaction between CD47 (e.g., hCD47) and SIRPα (e.g., hSIRPα) and the KRAS inhibitor (e.g., the KRAS G12C inhibitor or the KRAS G12D inhibitor) can be combined with supplemental doses of the agent that blocks the interaction between CD47 (e.g., hCD47) and SIRPα (e.g., hSIRPα) and/or the KRAS inhibitor. In some embodiments, the agent that blocks the interaction between CD47 (e.g., hCD47) and SIRPα (e.g., hSIRPα) and the KRAS inhibitor (e.g., the KRAS G12C inhibitor or the KRAS G12D inhibitor) are administered sequentially. In some embodiments, “sequential administration” means that the agent that blocks the interaction between CD47 (e.g., hCD47) and SIRPα. (e.g., hSIRPα) and the KRAS inhibitor (e.g., the KRAS G12C inhibitor or the KRAS G12D inhibitor) are administered with a time separation of more than about 15 minutes, such as more than about any of 20, 30, 40, 50, 60 or more minutes. For example, in some embodiments, the agent that blocks the interaction between CD47 and SIRPα is administered prior to the small molecule KRAS inhibitor (e.g., the KRAS G12C inhibitor or the KRAS G12D inhibitor). In some embodiments, the small molecule KRAS inhibitor is administered prior to the agent that blocks the interaction between CD47 and SIRPα. In some embodiments, the administration of the agent that blocks the interaction between CD47 and SIRPα and the KRAS inhibitor (e.g., the KRAS G12C inhibitor or the KRAS G12D inhibitor) are concurrent, i.e., the administration period of agent that blocks the interaction between CD47 and SIRPα and the KRAS inhibitor overlap with each other. In some embodiments, the administration of the agent that blocks the interaction between CD47 and SIRPα and the KRAS inhibitor (e.g., the KRAS G12C inhibitor or the KRAS G12D inhibitor) are non-concurrent.

### Exemplary Anti-CD47 Antibodies

An anti-CD47 antibody (or an immunologically active fragment thereof) is an antibody that binds to CD47 (e.g., human CD47 or “hCD47”) with sufficient affinity and specificity. As used herein, an “immunologically active fragment” of an antibody refers to an antigen-binding fragment of said antibody. The terms “immunologically active fragment” and “antigen-binding fragment” are used interchangeably herein. In some embodiments, the anti-CD47 antibody (or immunologically active fragment thereof) is a chimeric (such as humanized) monoclonal antibody (or immunologically active fragment thereof). In some embodiments, the anti-CD47 antibody is 5F9 (also known as Hu5F9-G4 and magrolimab), which is under development by Gilead Sciences/Forty Seven, Inc.; CC-90002 (also known as INBRX103), which is under development by Celgene; LQ001, which is under development by Novamab; HLX24, which is under development by Henlius; TI-061, which is under development by Arch Oncology (formerly Tioma Therapeutics); AO-176, which is under development by Arch Oncology; SRF-231, which is under development by Surface Oncology; IBI-188, which is under development by Innovent Bio; AKI 17, which is under development by Akesobio; IMC-002, which is under development by ImmuneOncia Therapeutics 3D Medicines; SHR-1603, which is under development by Jiangsu HengRui Medicine; STI-6643, which is under development by Sorrento Therapeutics Inc.; or ZL-1201, which is under development by Zai Lab. In some embodiments, the immunologically active fragment of the anti-CD47 antibody is an immunologically active fragment of any one of the preceding anti-CD47 antibodies. Additional details about these exemplary anti-CD47 antibodies can be found in, e.g., Jiang et al. (2021) J Hematol Oncol 14:180 doi(dot)org/10(dot)1186/s13045-021-01197-w; WO 2011/143624A2, U.S. Pat. No. 9,382,320 B2.

In some embodiments, the anti-CD47 antibody (or immunologically active fragment thereof) comprises a heavy chain variable domain (V_{H}), and/or a light chain variable domain (V_{L}) as described herein below.

In some embodiments, the anti-CD47 antibody (or immunologically active fragment thereof) comprises a (a) VH domain that comprises (1) a CDR-H1 comprising RAWMN (SEQ ID NO: 5); (2) a CDR-H2 comprising RIKRKTDGETTDYAAPVKG (SEQ ID NO: 6); (3) a CDR-H3 comprising SNRAFDI (SEQ ID NO: 7) and (b) a VL domain that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10). In some embodiments, the CDR sequences are defined according to Kabat (see, e.g., Kabat et al., Sequences of Proteins of Immunological Interest, 5th Ed. Public Health Service, National Institutes of Health, Bethesda, Md. (1991)). In some embodiments, the anti-CD47 antibody (or immunologically active fragment thereof) comprises a (a) VH domain that comprises (1) a CDR-H1 comprising GLTFERA (SEQ ID NO: 11); (2) a CDR-H2 comprising KRKTDGET (SEQ ID NO: 12); (3) a CDR-H3 comprising SNRAFDI (SEQ ID NO: 7) and (b) a VL domain that comprises (1) a CDR-L1 comprising KSSQSVLYAGNNRNYLA (SEQ ID NO: 14); (2) a CDR-L2 comprising QASTRAS (SEQ ID NO: 15); and (3) a CDR-L3 comprising QQYYTPPLA (SEQ ID NO: 16). In some embodiments, the CDR sequences are defined according to the Chothia numbering system (see, e.g., Chothia and Lesk (1986) EMBO J. 5(4):823-6 and Al-Lazikani et al., (1997) JMB 273: 927-948). In some embodiments, the anti-CD47 antibody (or immunologically active fragment thereof) comprises a (a) VH domain that comprises (1) a CDR-H1 comprising GLTFERAW (SEQ ID NO: 17); (2) a CDR-H2 comprising IKRKTDGETT (SEQ ID NO: 18); (3) a CDR-H3 comprising AGSNRAFDI (SEQ ID NO: 19) and (b) a VL domain that comprises (1) a CDR-L1 comprising QSVLYAGNNRNY (SEQ ID NO: 20); (2) a CDR-L2 comprising QAS (SEQ ID NO: 21); and (3) a CDR-L3 comprising QQYYTPPLA (SEQ ID NO: 22). In some embodiments, the CDR sequences are defined according to the IMGT numbering system (see, e.g., Lefranc M P. (2013) IMGT Unique Numbering. In: Dubitzky W., Wolkenhauer O., Cho K H., Yokota H. (eds) Encyclopedia of Systems Biology. Springer, New York, NY; https://doi.org/10.1007/978-1-4419-9863-7_127). In some embodiments, the anti-CD47 antibody (or immunologically active fragment thereof) comprises a (a) VH domain that comprises (1) a CDR-H1 comprising GLTFERAWMN (SEQ ID NO: 23); (2) a CDR-H2 comprising RIKRKTDGETTD (SEQ ID NO: 24); (3) a CDR-H3 comprising SNRAFDI (SEQ ID NO: 25) and (b) a VL domain that comprises (1) a CDR-L1 comprising KSSQSVLYAGNNRNYLA (SEQ ID NO: 26); (2) a CDR-L2 comprising QASTRAS (SEQ ID NO: 27); and (3) a CDR-L3 comprising QQYYTPPLA (SEQ ID NO: 28). In some embodiments, the CDR sequences are defined according to the AbM numbering system (see, e.g., Abhinandan R. K., Martin A. C. Analysis and improvements to Kabat and structurally correct numbering of antibody variable domains. Mol. Immunol. 2008; 45:3832-3839. doi: 10.1016/j.molimm.2008.05.022). In some embodiments, the anti-CD47 antibody (or immunologically active fragment thereof) comprises (a) VH domain that comprises (1) a CDR-H1 comprising ERAWMN (SEQ ID NO: 29); (2) a CDR-H2 comprising WVGRIKRKTDGETTD (SEQ ID NO: 30); (3) a CDR-H3 comprising AGSNRAFD (SEQ ID NO: 31) and (b) a VL domain that comprises (1) a CDR-L1 comprising LYAGNNRNYLAWY (SEQ ID NO: 32); (2) a CDR-L2 comprising LLINQASTRA (SEQ ID NO: 33); and (3) a CDR-L3 comprising QQYYTPPL (SEQ ID NO: 34). In some embodiments, the CDR sequences are defined according to the Contact numbering system (see, e.g., McCallum et al. (1996) J Mol Biol. 262(5):732-45; doi: 10.1006/jmbi.1996.0548).

For ease of reference, the amino acid sequences of SEQ ID NOs: 5-34 and are provided in Table A below.

TABLE A

| SEQ | | |
|---|---|---|
| ID | | |
| NO. | SEQUENCE | DESCRIPTION |
| 5 | RAWMN | HCDR1 of TJC4 (Kabat) |
| 6 | RIKRKTDGETTDYAAPVKG | HCDR2 of TJC4 (Kabat) |
| 7 | SNRAFDI | HCDR3 of TJC4 (Kabat) |
| 8 | KSSQSVLYAGNNRNYLA | LCDR1 of TJC4 (Kabat) |
| 9 | QASTRAS | LCDR2 of TJC4 (Kabat) |
| 10 | QQYYTPPLA | LCDR3 of TJC4 (Kabat) |
| 11 | GLTFERA | CDR-H1 of TJC4 (Chothia) |
| 12 | KRKTDGET | CDR-H2 of TJC4 (Chothia) |
| 13 | SNRAFDI | CDR-H3 of TJC4 (Chothia) |
| 14 | KSSQSVLYAGNNRNYLA | CDR-L1 of TJC4 (Chothia) |
| 15 | QASTRAS | CDR-L2 of TJC4 (Chothia) |
| 16 | QQYYTPPLA | CDR-L3 of TJC4 (Chothia) |
| 17 | GLTFERAW | CDR-H1 of TJC4 (IMGT) |
| 18 | IKRKTDGETT | CDR-H2 of TJC4 (IMGT) |
| 19 | AGSNRAFDI | CDR-H3 of TJC4 (IMGT) |
| 20 | QSVLYAGNNRNY | CDR-L1 of TJC4 (IMGT) |
| 21 | QAS | CDR-L2 of TJC4 (IMGT) |
| 22 | QQYYTPPLA | CDR-L3 of TJC4 (IMGT) |
| 23 | GLTFERAWMN | CDR-H1 of TJC4 (AbM) |
| 24 | RIKRKTDGETTD | CDR-H2 of TJC4 (AbM) |
| 25 | SNRAFDI | CDR-H3 of TJC4 (AbM) |
| 26 | KSSQSVLYAGNNRNYLA | CDR-L1 of TJC4 (AbM) |
| 27 | QASTRAS | CDR-L2 of TJC4 (AbM) |
| 28 | QQYYTPPLA | CDR-L3 of TJC4 (AbM) |
| 29 | ERAWMN | CDR-H1 of TJC4 (Contact) |
| 30 | WVGRIKRKTDGETTD | CDR-H2 of TJC4 (Contact) |
| 31 | AGSNRAFD | CDR-H3 of TJC4 (Contact) |
| 32 | LYAGNNRNYLAWY | CDR-L1 of TJC4 (Contact) |
| 33 | LLINQASTRA | CDR-L2 of TJC4 (Contact) |
| 34 | QQYYTPPL | CDR-L3 of TJC4 (Contact) |

In some embodiments, the anti-CD47 antibody (or immunologically active fragment thereof) comprises 3 CDRs of a VH domain comprising SEQ ID NO: 1. Additionally or alternatively, in some embodiments, the anti-CD47 antibody (or immunologically active fragment thereof) comprises 3 CDRs of a VL domain comprising SEQ ID NO: 2. In some embodiments, the 3 CDRs of the VH domain are CDRs according to Kabat, Chothia, AbM or Contact numbering system. Additionally or alternatively, in some embodiments, the 3 CDRs of the VL domain are CDRs according to Kabat, Chothia, AbM or Contact numbering system. In some embodiments, the N-terminal amino acid of the VH domain of the anti-CD47 antibody (or immunologically active fragment thereof) is E, and, optionally, in some embodiments, the C-terminal amino acid of the VH domain of the anti-CD47 antibody (or immunologically active fragment thereof) is S.

In some embodiments, the anti-CD47 antibody (or immunologically active fragment thereof) comprises a heavy chain variable domain (VH) comprising an amino acid sequence that has at least about 95%, 96%, 97%, 98%, 99%, or 100% sequence identity to an amino acid sequence set forth in SEQ ID NO: 1. In some embodiments, the N-terminal amino acid of the VH domain that has at least about 95%, 96%, 97%, 98%, 99%, or 100% sequence identity to an amino acid sequence set forth in SEQ ID NO: 1 is E. Additionally or alternatively, in some embodiments, the C-terminal amino acid of the VH domain that has at least about 95%, 96%, 97%, 98%, 99%, or 100% sequence identity to an amino acid sequence set forth in SEQ ID NO: 1 is S. In some embodiments, the N-terminal amino acid of the V_{H} domain of the anti-CD47 antibody (or immunologically active fragment thereof) corresponds to position H1 according to the Kabat numbering system, and the C-terminal amino acid of the V_{H} domain of the anti-CD47 antibody (or immunologically active fragment thereof) corresponds to position H113 according to the Kabat numbering system. In some embodiments, the N-terminal amino acid of the V_{H} domain of the anti-CD47 antibody (or immunologically active fragment thereof) corresponds to position H1 according to the Chothia numbering system, and the C-terminal amino acid of the V_{H} domain of the anti-CD47 antibody (or immunologically active fragment thereof) corresponds to position H113 according to the Chothia numbering system. In some embodiments, the N-terminal amino acid of the V_{H} domain of the anti-CD47 antibody (or immunologically active fragment thereof) corresponds to position H1 according to the IMGT numbering system, and the C-terminal amino acid of the V_{H} domain of the anti-CD47 antibody (or immunologically active fragment thereof) corresponds to position H128 according to the IMGT numbering system. In some embodiments, the N-terminal amino acid of the V_{H} domain of the anti-CD47 antibody (or immunologically active fragment thereof) corresponds to amino acid 1 of SEQ ID NO: 1, and the C-terminal amino acid of the V_{H} domain the anti-CD47 antibody (or immunologically active fragment thereof) corresponds to amino acid 118 of SEQ ID NO: 1. In some embodiments, the anti-CD47 antibody comprises (such as further comprises) a light chain variable domain (VL) comprising an amino acid sequence that has at least about 95%, 96%, 97%, 98%, 99%, or 100% sequence identity to an amino acid sequence set forth in SEQ ID NO: 2. In some embodiments, the anti-CD47 antibody comprises a VH comprising SEQ ID NO: 1 and a VL comprising SEQ ID NO: 2. The amino acid sequences of SEQ ID NOs: 1 and 2 are provided below:

(SEQ ID NO: 1)
EVQLVESGGG LVKPGGSLRL SCAASGLTFE RAWMNWVRQA
PGKGLEWVGR IKRKTDGETT DYAAPVKGRF SISRDDSKNT
LYLQMNSLKT EDTAVYYCAG SNRAFDIWGQ GTMVTVSS
(SEQ ID NO: 2)
DIVMTQSPDS LAVSLGERAT INCKSSQSVL YAGNNRNYLA
WYQQKPGQPP KLLINQASTR ASGVPDRFSG SGSGTEFTLI
ISSLQAEDVA IYYCQQYYTP PLAFGGGTKL EIK

In some embodiments, the anti-CD47 antibody is a full-length antibody. In some embodiments, the full-length antibody comprises a human Fc region. In some embodiments, the human Fc region is an IgG1, IgG2, or IgG4 Fc region. In some embodiments, the full length anti-CD47 antibody comprises a human IgG4 Fc region or a variant thereof that comprises an S228P substitution, wherein amino acid numbering is according to the EU index. In some embodiments, the full length anti-CD47 antibody comprises a heavy chain comprising the amino acid SEQ ID NO: 3 and a light chain comprising the amino acid sequence of SEQ ID NO: 4. In some embodiments, the full length anti-CD47 antibody is a full length antibody that comprises a heavy chain comprising the amino acid SEQ ID NO: 35 and a light chain comprising the amino acid sequence of SEQ ID NO: 4. In some embodiments, the anti-CD47 antibody is lemzoparlimab (also known as TJ011133).

(SEQ ID NO: 3)
EVQLVESGGG LVKPGGSLRL SCAASGLTFE RAWMNWVRQA
PGKGLEWVGR IKRKTDGETT DYAAPVKGRF SISRDDSKNT
LYLQMNSLKT EDTAVYYCAG SNRAFDIWGQ GTMVTVSSAS
TKGPSVFPLA PCSRSTSEST AALGCLVKDY FPEPVTVSWN
SGALTSGVHT FPAVLQSSGL YSLSSVVTVP SSSLGTKTYT
CNVDHKPSNT KVDKRVESKY GPPCPPCPAP EFLGGPSVFL
FPPKPKDTLM ISRTPEVTCV VVDVSQEDPE VQFNWYVDGV
EVHNAKTKPR EEQFNSTYRV VSVLTVLHQD WINGKEYKCK
VSNKGLPSSI EKTISKAKGQ PREPQVYTLP PSQEEMTKNQ
VSLTCLVKGF YPSDIAVEWE SNGQPENNYK TTPPVLDSDG
SEFLYSRLTV DKSRWQEGNV FSCSVMHEAL HNHYTQKSLS
LSLGK
(SEQ ID NO: 35)
EVQLVESGGG LVKPGGSLRL SCAASGLTFE RAWMNWVRQA
PGKGLEWVGR IKRKTDGETT DYAAPVKGRF SISRDDSKNT
LYLQMNSLKT EDTAVYYCAG SNRAFDIWGQ GTMVTVSSAS
TKGPSVFPLA PCSRSTSEST AALGCLVKDY FPEPVTVSWN
SGALTSGVHT FPAVLQSSGL YSLSSVVTVP SSSLGTKTYT
CNVDHKPSNT KVDKRVESKY GPPCPPCPAP EFLGGPSVFL
FPPKPKDTLM ISRTPEVTCV VVDVSQEDPE VQFNWYVDGV
EVHNAKTKPR EEQFNSTYRV VSVLTVLHQD WLNGKEYKCK
VSNKGLPSSI EKTISKAKGQ PREPQVYTLP PSQEEMTKNQ
VSLTCLVKGF YPSDIAVEWE SNGQPENNYK TTPPVLDSDG
SFFLYSRLTV DKSRWQEGNV FSCSVMHEAL HNHYTQKSLS
LSLG
(SEQ ID NO: 4)
DIVMTQSPDS LAVSLGERAT INCKSSQSVL YAGNNRNYLA
WYQQKPGOPP KLLINQASTR ASGVPDRFSG SGSGTEFTLI
ISSLQAEDVA IYYCQQYYTP PLAFGGGTKL EIKRTVAAPS
VFIFPPSDEQ LKSGTASVVC LLNNFYPREA KVQWKVDNAL
QSGNSQESVT EQDSKDSTYS LSSTLTLSKA DYEKHKVYAC
EVTHQGLSSP VTKSFNRGEC

The anti-CD47 antibody that binds specifically to hCD47 can be of any of the various types of antibodies as defined above, but is, in certain embodiments, a human, humanized, or chimeric antibody. In some embodiments, the anti-CD47 antibody is a human antibody. In some embodiments, the anti-CD47 is a humanized antibody that comprises a human antibody constant domain (e.g., a human Fc domain, such as a human IgG Fc domain, e.g., a human IgG1, a human IgG2, a human IgG3, or a human IgG4 Fc domain, or a variant of a human IgG4 Fc domain that comprises an S228P substitution, wherein amino acid numbering is according to the EU index). In some embodiments, the anti-CD47 antibody is a chimeric antibody. See, e.g., U.S. Pat. No. 4,816,567 and Morrison et al., Proc. Natl. Acad. Sci. USA, 81:6851-6855 (1984). In some embodiments, the chimeric anti-CD47 antibody comprises a non-human variable region (e.g., a variable region derived from a chicken, mouse, rat, hamster, rabbit, or non-human primate, such as a monkey) and a human constant region. In some embodiments, a chimeric antibody is a “class switched” antibody in which the class or subclass has been changed from that of the parent antibody. Chimeric antibodies include antigen-binding fragments thereof.

In some embodiments, a chimeric antibody is a humanized antibody. A non-human antibody can be humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody (e.g., a chicken antibody), and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR or CDR residues are derived), e.g., to restore or improve antibody specificity or affinity. Humanized antibodies and methods of making them are reviewed, e.g., in Almagro and Fransson, Front. Biosci. 13:1619-1633 (2008).

Human framework regions useful for humanization include but are not limited to: framework regions selected using the “best-fit” method; framework regions derived from the consensus sequence of human antibodies of a particular subgroup of light or heavy chain variable regions; human somatically mutated framework regions or human germline framework regions; and framework regions derived from screening FR libraries. See, e.g., Sims et al. J. Immunol. 151:2296 (1993); Carter et al. Proc. Natl. Acad. Sci. USA, 89:4285 (1992); Presta et al. J. Immunol., 151:2623 (1993); Almagro and Fransson, Front. Biosci. 13:1619-1633 (2008); and Baca et al., J. Biol. Chem. 272:10678-10684 (1997).

In some embodiments, an anti-CD47 antibody of the present disclosure is a human antibody. Human antibodies can be produced using various techniques known in the art. In some embodiments, the human antibody is produced by a non-human animal, such as the genetically engineered chickens (see, e.g., U.S. Pat. Nos. 8,592,644; and 9,380,769) and/or mice described herein. Human antibodies are described generally in Lonberg, Curr. Opin. Immunol. 20:450-459 (2008).

In some embodiments, an anti-CD47 antibody of the present disclosure is an antibody fragment (e.g., an immunologically active fragment), including without limitation a Fab, F(ab′)₂, Fab′-SH, Fv, or scFv fragment, or a single domain, single heavy chain, or single light chain antibody. As used herein, an “immunologically active fragment” of an antibody refers to an antigen-binding fragment of said antibody. The terms “immunologically active fragment” and “antigen-binding fragment” are used interchangeably herein. Antibody fragments can be generated, e.g., by enzymatic digestion or by recombinant techniques. In some embodiments, Proteolytic digestion of an intact antibody is used to generate an antibody fragment, e.g., as described in Morimoto et al., Journal of Biochemical and Biophysical Methods 24:107-117 (1992) and Brennan et al., Science, 229:81 (1985). In some embodiments, an antibody fragment is produced by a recombinant host cell. For example, Fab, Fv and ScFv antibody fragments are expressed by and secreted from E. coli. Antibody fragments can alternatively be isolated from an antibody phage library. Other methods of generating immunologically active fragments of an antibody are well-known in the art.

In some embodiments, the anti-CD47 antibody (or immunologically active fragment thereof) specifically recognizes (such as binds) to hCD47 expressed on the surface of a cell. In some embodiments, the anti-CD47 antibody specifically recognizes hCD47 expressed on the surface of a cancer cell, e.g., a KRAS G12C mutant cancer cell or a KRAS G12D mutant cancer cell (such as a lung adenocarcinoma cell, a non-small cell lung cancer (NSCLC) cell, a colon adenocarcinoma cell, a colorectal adenocarcinoma cell, a pancreatic cancer cell, a cholangiocarcinoma cell, an endometrial cancer cell, an ovarian cancer cell, a peritoneal cancer cell, a bladder cancer cell, a gastric cancer cell, a thyroid cancer cell, a melanoma cell, a breast cancer cell, a head and neck cancer cell, a multiple myeloma cell, an acute myeloid leukemia (AML) cell, a uterine cancer cell, a gastro-esophageal cancer cell, or a rectal carcinoma cell). In some embodiments, the binding of an anti-CD47 antibody (or immunologically active fragment thereof) described herein to hCD47 (e.g., hCD47 expressed on the surface of a cell) prevents the interaction of hCD47 with signal regulatory protein alpha (SIRPα), such as human SIRPα (“hSIRPα”). In some embodiments, the binding of an anti-CD47 antibody (or immunologically active fragment thereof) described herein to hCD47 expressed on the surface of a cancer cell (e.g., a KRAS G12C mutant cancer cell or a KRAS G12D mutant cancer cell) promotes macrophage mediated phagocytosis of the cancer cell.

Further details about exemplary anti-CD47 antibodies that may be used with the methods of treating a KRAS mutant cancer (e.g., a KRAS G12C mutant cancer or a KRAS G12D mutant cancer), including pharmaceutical formulations comprising same, exemplary dosages, and exemplary administration schedules, are provided in PCT/CN2021/123892. Further details about methods of producing/manufacturing anti-CD47 antibodies described herein are provided in PCT/CN2021/123892.

### KRAS and Exemplary KRAS Inhibitors

KRAS, a member of the RAS family, is a key regulator of signaling pathways responsible for cell proliferation, differentiation, and survival. See, e.g., Cox et al. (2003) Nat Rev Drug Discov. 13(11):828-851 and Downward J. (2003) Nat Rev Cancer. 3(1):11-22. KRAS is the most frequently mutated oncogene in human cancer, and mutations in KRAS can result in continuous cellular proliferation and cancer development. The KRAS G12C mutation occurs in about 13% of NSCLC patients, and 1%-3% of colorectal and other solid tumors. G12C is a single point mutation with a glycine-to-cysteine substitution at codon 12. See Cox et al. (2003) Nat Rev Drug Discov. 13(11):828-851; Neumann et al. (2009) Pathol Res Pract. 205:858-862; and Biernacka et al. (2016) Cancer Genet. 209(5):195-198. This substitution favors the activated GTP-bound state of KRAS, amplifying signaling pathways that lead to oncogenesis (see Ryan et al. (2018) Nat Rev Clin Oncol. 15(11):709-720 and Kim et al. (2020 Cell, 183(4): 850-859). The KRAS G12D mutation occurs in about one in three pancreatic cancers and in about one in ten colorectal cancers. The mutation of glycine at position 12 to aspartate (G12D) leads to the projection of a bulkier and negatively charged side group into the active site, which causes a steric hindrance in GTP hydrolysis (see Malumbres et al. (2003) Nat Rev Cancer, 3, 459-465), impairs the GTPase function, and locks KRAS in its active (GTP-bound) state (see Scheffzek et al. (1997) Science 277, 333-338).

In some embodiments, the KRAS inhibitor is a KRAS G12C inhibitor, and the cancer (or population of cancer cells) comprises one or more cells that express the KRAS G12C mutant protein. In some embodiments, the KRAS G12C mutant cancer is lung cancer, colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12C mutant cancer is lung adenocarcinoma or non-small cell lung cancer (NSCLC).

In some embodiments, the term “KRAS G12C inhibitor” refers to any agent, e.g., polypeptide, fusion polypeptide, antibody, peptide, antisense oligonucleotide, or small molecule drug that inhibits the activity of the KRAS G12C mutant protein. In some embodiments, the KRAS G12C inhibitor interacts directly with the KRAS G12C mutant protein to inhibit the protein's activity. In some embodiments, the KRAS G12C inhibitor is a small molecule drug. Exemplary small molecule KRAS G12C inhibitors that find use with the methods provided herein include, without limitation, e.g., AMG 510 (also known as sotorasib, LUMAKRAS™, and LUMYKRAS™), MRTX849 (also known as adagrasib), JAB-21822 (also known as JAB-21000), GDC-6036, JDQ443, D-1553, GH35, GFH925, BPI-421286, or LY3537982. Other exemplary small molecule KRAS G12C inhibitors are described in, e.g., Hillig et al. (2019) Proc Natl Acad Sci USA. 116(7): 2551-2560 and Sun et al. (2012) Angew Chem Int Ed Engl. 51(25): 6140-6143, and in WO 2020/233592, WO 2021/023247, WO 2021/083167, WO 2020/238791, WO 2021/000885, CN 112585129, CN 112552295, CN 112390818, CN 112390796, WO 2019/141250, CN 111592528, WO 2020/259432, CN 112300153, CN 112552294, WO 2020/239123, CN 110698378, CN 111377918, CN 111205286, CN 112047939, WO 2020/259513, WO 2021/023154, CN 112430234, WO 2021/063346, WO 2021/058018, CN 112225734, WO 2021/155716, CN112159405, CN 112778302, CN 112830928, WO 2020/1027943, CN 112574199, WO 2021/037018, CN 110172089, WO 2020/156285, CN 111499634, WO 2020/177629, WO 2020/216190. WO 2020/221239, WO 2020/233592, WO 2020/238791, WO 2020/239077, WO 2020/239123, CN 112047933, CN 112047937, CN 112047948, WO 2020/259513, WO 2020/259573, WO 2020/259432, WO 2021/000885, CN 112174950, CN 112300153, WO 2021/023154, WO 2021/023247, CN 112390818, WO 2021/027943, WO 2021/027911, WO 2021/031952, CN 112390796, WO 2021/037018, CN 112442029, WO 2021/043322, WO 2021/052499, CN 112538084. CN 112552295, WO 2021/058018, WO 2021/063346, WO 2021/068898, WO 2021/078285, CN 112225734, CN 112707905, CN 112745335, WO 2021/083167, CN 112778284, WO 2021/088458, CN 112851663, WO 2021/093758, WO 2021/098859, CN 112830928, CN 111377918, WO 2021/104431, WO 2021/109737, WO 2021/113595, CN 112920183, WO 2021/121371, WO 2021/121367, CN 113004269, WO 2021/129824, WO 2021/129820, CN 113061132, WO 2021/139678, WO 2021/139748, CN 111205286, WO 2021/143693, CN 113135924, WO 2021/147965, WO 2021/155716, WO 2021/168193, WO 2021/169990, WO 2021/169963, CN 113321654, WO 2021/175199, WO 2021/180181, WO 2021/185233, WO 2021/190467, WO 2021/197499, CN 112574199 and CN 112300269, the contents of which are incorporated herein by reference in their entireties.

In some embodiments, the KRAS G12C inhibitor is AMG 510, which, as noted above, is also known as sotorasib, LUMAKRAS™, and LUMYKRAS™. AMG 510 is currently under development by Amgen/Beigene. AMG 510 can exist in either of two atropisomeric forms and one is more active than the other (see, e.g., https://cen(dot)acs(dot)org/pharmaceuticals/drug(dash)discovery/Amgen(dash)unveils(dash)KRa s(dash)inhibitor(dash)human/97/i14). AMG 510 selectively forms an irreversible covalent bond to the sulfur atom in the cysteine residue that is present in the G12C mutated form of the KRAS protein, but not in the wild type form. The covalent binding of AMG 150 to KRAS G12C locks the protein in its inactive GDP-bound conformation, thus inhibiting KRAS-dependent signal transduction. AMG 150 has the empirical formula C₃₀HF₂N₆O₃ and a molecular weight of 560.606 g/mol. AMG 150 is described chemically as 6-Fluoro-7-(2-fluoro-6-hydroxyphenyl)-(1M)-1-[4-methyl-2-(propan-2-yl)pyridin-3-yl]-4-[(2S)-2-methyl-4-(prop-2-enoyl)piperazin-1-yl]pyrido[2,3-d]pyrimidin-2(1H)-one and has the following chemical structure:

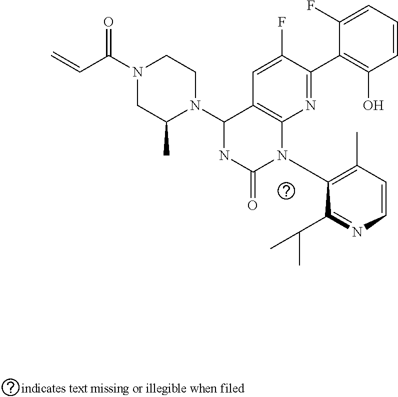

The CAS Registry Number for AMG 510 is 2252403-56-6. The efficacy of AMG 510 was demonstrated in a subset of patients enrolled in a single-arm, open-label, multicenter trial (NCT03600883) and is currently being investigated in further clinical trials. Complete information about AMG 510 preparation, dispensing, dosage, and administration schedule can be found in the local package insert (for the United States, see, e.g., www(dot)accessdata(dot)fda(dot)gov/drugsatfda_docs/label/2021/214665s0001bl.pdf. Further details regarding the structure and synthesis of AMG 510 are provided in WO 2018/217651, the contents of which are incorporated herein by reference in their entirety.

In some embodiments, the KRAS G12C inhibitor is MRTX849 (also known as adagrasib). MRTX849 is currently under development by Mirati/Zai Lab. MRTX849 selectively forms an irreversible covalent bond to the sulfur atom in the cysteine residue that is present in the G12C mutated form of the KRAS protein, but not in the wild type form. The covalent binding of MRTX849 to KRAS G12C locks the protein in its inactive GDP-bound conformation, thus inhibiting KRAS-dependent signal transduction. MRTX849 has the empirical formula C₃₂H₃₅ClFN₇O₂ and a molecular weight of 604.13 g/mol. MRTX849 is described chemically as 2-[(2S)-4-[7-(8-chloronaphthalen-1-yl)-2-[[(2S)-1-methylpyrrolidin-2-yl]methoxy]-6,8-dihydro-5H-pyrido[3,4-d]pyrimidin-4-yl]-1-(2-fluoroprop-2-enoyl)piperazin-2-yl]acetonitrile and has the following chemical structure:

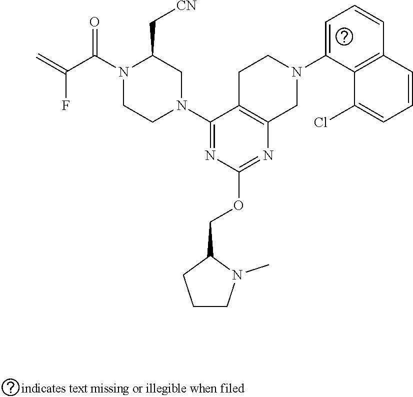

The CAS Registry Number for MRTX849 is 2326521-71-3. MRTX849 is currently being evaluated in several clinical trials, including NCT04613596, NCT04685135, NCT03785249, NCT04330664, and others. Further details regarding the structure and synthesis of MRTX849 are provided in Fell et al. (2020) J. Med. Chem. 63, 6679-6693 and WO 2017/201161, the contents of which are incorporated herein by reference in their entirety.

In some embodiments, the KRAS G12C inhibitor is JAB-21822. JAB-21822 is under development by Jacobio Pharmaceuticals Group Co., LTD. (see, e.g., en(dot)jacobiopharma(dot)com/blank3(dot)html?introId=23 and www(dot)jacobiopharma(dot)com/news/207(dot)html). JAB-21822 is currently being evaluated in several clinical trials, including NCT05009329 and NCT05002270. Further details regarding the structure and synthesis of JAB-21822 are provided in WO 2021/057832, the contents of which are incorporated herein by reference in their entirety.

In some embodiments, the KRAS G12C inhibitor is GDC-6036. GDC-6036 is under development by Genentech, Inc. (see, e.g., www(dot)genentechoncology(dot)com/pipeline-molecules/kras-g12c.html) and is currently being evaluated clinical trial NCT04449874. Further details regarding the structure and synthesis of GDC-6036 are provided in WO 2020/097537, the contents of which are incorporated herein by reference in their entirety.

In some embodiments, the KRAS G12C inhibitor is JDQ443. JDQ443 is under development by Novartis. The structure of JDQ443 is:

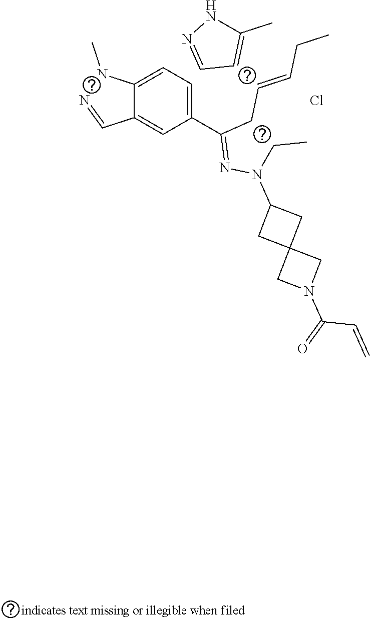

JDQ443 is currently being evaluated in clinical trial NCT04699188. Further details regarding the synthesis of JDQ443 are provided in WO 2021/124222, the contents of which are incorporated herein by reference in their entirety.

In some embodiments, the KRAS G12C inhibitor is D-1553. D-1553 is under development by InventisBio Co., Ltd. (see, e.g., www(dot)inventisbio(dot)com/%e4%b8%b4%e5%ba%8a%e8%af%95%e9%aa%8c/) and is currently being evaluated in clinical trial NCT04585035 in collaboration with Merck Sharp & Dohme. Further details regarding the structure and synthesis of D-1553 are provided in WO 2020/233592, the contents of which are incorporated herein by reference in their entirety.

In some embodiments, the KRAS G12C inhibitor is GH35. GH35 is under development by Suzhou Genhouse Bio Co., Ltd. (see, e.g., www(dot)genhousebio(dot)com/en/product/index(dot)html) and is being evaluated in clinical trial NCT05010694. Further details regarding the structure and synthesis of GH35 are provided in WO2020/177653, the contents of which are incorporated herein by reference in their entirety.

In some embodiments, the KRAS G12C inhibitor is GFH925. GFH925 is under development by GenFleet Therapeutics (Zhejiang) (see, e.g., www(dot)genfleet(dot)com/en/science) and is being evaluated in clinical trial NCT05005234. Further details regarding the structure and synthesis of GFH925 are provided in WO 2020/177629, WO 2020/221239, and WO 2021/031952, the contents of which are incorporated herein by reference in their entirety.

In some embodiments, the KRAS G12C inhibitor is BPI-421286. BPI-421286 is under development by Betta Pharmaceutical Co., Ltd (see, e.g., www(dot)bettapharma(dot)com/News/show/id/2380), and clinical trial applications for the evaluation of BPI-421286 (CXHL2100046 and CXHL2100047) have been accepted by the State Food and Drug Administration of the People's Republic of China. Further details regarding the structure and synthesis of BPI-421286 are provided in CN112390796, the contents of which are incorporated herein by reference in their entirety.

In some embodiments, the KRAS G12C inhibitor is LY3537982. LY3537982 is under development by Eli Lilly and Company and Loxo Oncology, Inc. (see, e.g., www(dot)lillyloxooncologypipeline(dot)com/molecule/kras-g12c-inhibitor/) and is being investigated in clinical trial NCT04956640. Further details regarding the structure and synthesis of LY3537982 are provided in WO2021/118877, the contents of which are incorporated herein by reference in their entirety.

In some embodiments, the KRAS inhibitor is a KRAS G12D inhibitor, and the cancer (or population of cancer cells) comprises one or more cells that express the KRAS G12D mutant protein. In some embodiments, the KRAS G12D mutant cancer is lung cancer, colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12D mutant cancer is colorectal cancer, such as colorectal adenocarcinoma, or colon cancer, such as colon adenocarcinoma. In some embodiments, the term “KRAS G12D inhibitor” refers to any agent, e.g., polypeptide, fusion polypeptide, antibody, peptide, antisense oligonucleotide, or small molecule drug that inhibits the activity of the KRAS G12D mutant protein. In some embodiments, the KRAS G12D inhibitor interacts directly with the KRAS G12D mutant protein to inhibit the protein's activity. In some embodiments, the KRAS G12D inhibitor is a small molecule drug. An exemplary small molecule KRAS G12D inhibitor that finds use with the methods provided herein is MRTX1133. In some embodiments, the KRAS G12D inhibitor that finds use with the methods provided herein is or comprises an siRNA that targets an mRNA encoding the KRAS G12D mutant protein (also referred to herein as “a KRAS G12D siRNA.” In some embodiments, the KRAS G12D siRNA is loaded into mesenchymal stromal cell-derived exosomes, also known as “iExosomes” (see, e.g., clinical trial NCT03608631 and Surana et al. (2022) Journal of Clinical Oncology, 40:4_suppl, TPS633-TPS633).

In some embodiments, the KRAS G12D inhibitor is MRTX1133. MRTX1133, which is under development by Mirati Therapeutics, Inc., demonstrates selective and reversible inhibition of the KRAS G12D mutant protein in both its active and inactive states (Wang et al. (2022) J Med Chem, 65(4):3123-3133). The specificity of MRTX1133 to the KRAS G12D mutant protein is more than 1000 times of that of wild-type KRAS (Wang et al. (2022) J Med Chem, 65(4):3123-3133). MRTX1133 has the empirical formula C₃₃H₃₁F₃N₆O₂ and a molecular weight of 600.63 g/mol. MRTX1133 has the following chemical structure:

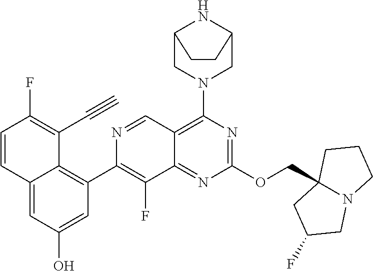 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]

The CAS Registry Number for MRTX1133 is 2621928-55-8. MRTX1133 is currently being evaluated in clinical trial NCT05737706. Further details regarding MRTX1133 are provided in, e.g., Wang et al. (2022) J Med Chem, 65(4):3123-313; Xie et al. (2021) Front Oncol, 11: 672612; and Hallin et al. (2022) Nat Med, 28(10):2171-2182.

In some embodiments, the KRAS G12D inhibitor is or comprises an siRNA that targets an mRNA encoding the KRAS G12D mutant protein (also referred to herein as “a KRAS G12D siRNA.” In some embodiments, the KRAS G12D siRNA is loaded into bone marrow mesenchymal/stromal cell-derived exosomes. Such exosomes are known as “iExosomes” (see, e.g., clinical trial NCT03608631 and Surana et al. (2022) Journal of Clinical Oncology, 40:4_suppl, TPS633-TPS633). Additional details regarding iExosomes are provided in Mendt et al. (2018) JCI Insight, 3(8):e99263 and Kamerkar et al. (2017) Nature. 546: 498-503.

### Exemplary Methods

In some embodiments, provided is a method of treating a KRAS mutant cancer (i.e., a cancer comprising one or more cancer cells that express a KRAS mutant protein, e.g., a KRAS G12C mutant protein or a KRAS G12D mutant protein) in a subject (e.g., a human subject), comprising administering to the subject an effective amount of an agent (e.g., therapeutic agent) that blocks the interaction between CD47 (e.g., hCD47) and SIRPα (e.g., hSIRPα) and an effective amount of a KRAS inhibitor (e.g., a KRAS G12C inhibitor wherein the cancer is a KRAS G12C mutant cancer, or a KRAS G12D inhibitor wherein the cancer is a KRAS G12C mutant cancer). In some embodiments, provided is a method of stimulating the phagocytosis of a population of cancer cells that comprises one or more cancer cells that express a KRAS mutant protein (e.g., a KRAS G12C mutant protein or a KRAS G12D mutant protein), comprising contacting the population with an effective amount of an agent (e.g., therapeutic agent) that blocks the interaction between CD47 (e.g., hCD47) and SIRPα (e.g., hSIRPα) and an effective amount of a KRAS inhibitor (e.g., a KRAS G12C inhibitor wherein the KRAS mutant protein is a KRAS G12C mutant protein, or a KRAS G12D inhibitor wherein the KRAS mutant protein is a KRAS G12D mutant protein). In some embodiments, the KRAS mutant cancer (e.g., the KRAS G12C mutant cancer or the KRAS G12D mutant cancer) or the one or more cancer cells that expresses KRAS mutant protein (e.g., a KRAS G12C mutant protein or the KRAS G12D mutant protein) is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS mutant cancer is a KRAS G12C mutant cancer. In some embodiments, the population of cancer cells comprises one or more cells that express the KRAS G12C mutant protein. In some embodiments, the KRAS G12C mutant cancer (or the one or more cancer cells that expresses KRAS G12C mutant protein) is NSCLC. In some embodiments, the KRAS mutant cancer is a KRAS G12D mutant cancer. In some embodiments, the population of cancer cells comprises one or more cells that express the KRAS G12D mutant protein. In some embodiments, the KRAS G12D mutant cancer (or the one or more cancer cells that expresses KRAS G12D mutant protein) is colorectal cancer, e.g., colorectal adenocarcinoma, or colon cancer, e.g., colon adenocarcinoma. In some embodiments of the methods of treatment, the agent that blocks the interaction between CD47 and SIRPα and the KRAS inhibitor (e.g., the KRAS G12C inhibitor or the KRAS G12D inhibitor) are administered simultaneously. In some embodiments of the methods of treatment, the agent that blocks the interaction between CD47 and SIRPα and the KRAS inhibitor (e.g., the KRAS G12C inhibitor or the KRAS G12D inhibitor) are administered sequentially. In some embodiments of the methods of treatment, the agent that blocks the interaction between CD47 and SIRPα is administered prior to the KRAS inhibitor. In some embodiments of the methods of treatment, the KRAS inhibitor is administered prior to the agent that blocks the interaction between CD47 and SIRPα. In some embodiments, the agent that blocks the interaction between CD47 and SIRPα is a polypeptide. In some embodiments, the agent that blocks the interaction between CD47 and SIRPα is an antibody, antibody construct or immunologically active fragment of the antibody or antibody construct. In some embodiments, the agent that blocks the interaction between CD47 and SIRPα is an anti-CD47 antibody (or immunologically active fragment thereof). In some embodiments, the KRAS inhibitor is a KRAS G12C inhibitor. In some embodiments, the KRAS G12C inhibitor is a small molecule inhibitor. In some embodiments, the KRAS G12C inhibitor is AMG 510, shown below:

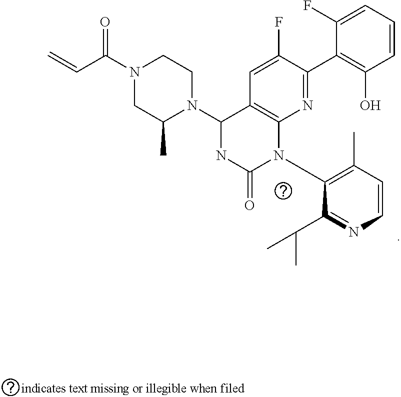

In some embodiments, the KRAS G12C inhibitor is MRTX849, shown below:

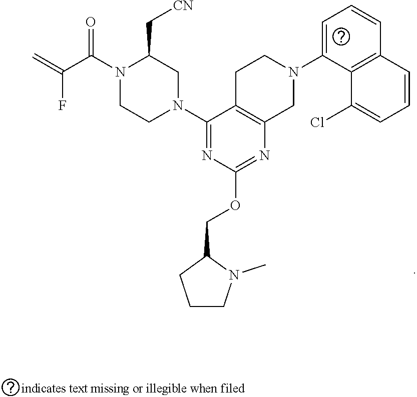

In some embodiments, the KRAS inhibitor is a KRAS G12D inhibitor. In some embodiments, the KRAS G12D inhibitor is a small molecule inhibitor. In some embodiments, the KRAS G12D inhibitor is MRTX1122, shown below:

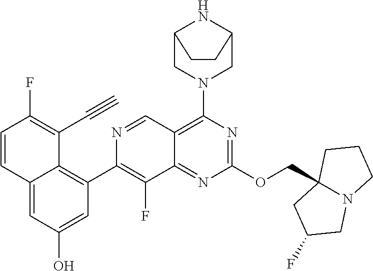 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]

In some embodiments, provided is a method of treating a KRAS mutant cancer (i.e., a cancer comprising one or more cancer cells that express a KRAS mutant protein, e.g., a KRAS G12C mutant protein or a KRAS G12D mutant protein) in a subject (e.g., a human subject), comprising administering to the subject an effective amount of an anti-CD47 antibody (or immunologically active fragment thereof) that blocks the interaction between CD47 (e.g., hCD47) and SIRPα (e.g., hSIRPα) and an effective amount of a KRAS G inhibitor (e.g., a KRAS G12C inhibitor wherein the cancer is a KRAS G12C mutant cancer, or a KRAS G12D inhibitor wherein the cancer is a KRAS G12C mutant cancer). In some embodiments, provided is a method of stimulating the phagocytosis of a population of cancer cells that comprises one or more cancer cells that express a KRAS G12C mutant protein, comprising contacting the population with an effective amount of an anti-CD47 antibody (or immunologically active fragment thereof) that blocks the interaction between CD47 (e.g., hCD47) and SIRPα (e.g., hSIRPα) and an effective amount of a KRAS G12C inhibitor. In some embodiments, the KRAS G12C mutant cancer (or the one or more cancer cells that expresses KRAS G12C mutant protein) is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12C mutant cancer (or the one or more cancer cells that expresses KRAS G12C mutant protein) is NSCLC. In some embodiments of the methods of treatment, the anti-CD47 antibody (or immunologically active fragment thereof) and the KRAS G12C inhibitor are administered simultaneously. In some embodiments of the methods of treatment, the anti-CD47 antibody (or immunologically active fragment thereof) and the KRAS G12C inhibitor are administered sequentially. In some embodiments of the methods of treatment, the anti-CD47 antibody (or immunologically active fragment thereof) is administered prior to the KRAS G12C inhibitor. In some embodiments of the methods of treatment, the KRAS G12C inhibitor is administered prior to the anti-CD47 antibody (or immunologically active fragment thereof). In some embodiments, the anti-CD47 antibody is CC-90002 (also known as INBRX103), 5F9 (also known as Hu5F9-G4 and magrolimab), LQ001, HLX24, TI-061, AO-176, SRF-231, IBI-188, IMC-002, SHR-1603, STI-6643, or ZL-1201, or an immunologically active fragment of any one of the preceding. In some embodiments, the anti-CD47 antibody (or immunologically active fragment thereof) is an anti-CD47 antibody (or immunologically active fragment thereof) described herein. In some embodiments, the KRAS G12C inhibitor is a small molecule inhibitor. In some embodiments, the KRAS G12C inhibitor is AMG 510, the structure of which is shown above. In some embodiments, the KRAS G12C inhibitor is MRTX849, the structure of which is shown above.

In some embodiments, provided is a method of treating a KRAS G12C mutant cancer (i.e., a cancer comprising one or more cancer cells that express a KRAS G12C mutant protein) in a subject (e.g., a human subject), comprising administering to the subject an effective amount of an anti-CD47 antibody (or immunologically active fragment thereof) that blocks the interaction between CD47 (e.g., hCD47) and SIRPα (e.g., hSIRPα) and an effective amount of a KRAS G12C inhibitor. In some embodiments, the KRAS G12C mutant cancer is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12C mutant cancer is NSCLC. In some embodiments of the methods of treatment, the anti-CD47 antibody (or immunologically active fragment thereof) and the KRAS G12C inhibitor are administered simultaneously. In some embodiments of the methods of treatment, the anti-CD47 antibody (or immunologically active fragment thereof) and the KRAS G12C inhibitor are administered sequentially. In some embodiments of the methods of treatment, the anti-CD47 antibody (or immunologically active fragment thereof) is administered prior to the KRAS G12C inhibitor. In some embodiments, the KRAS G12C inhibitor is administered prior to the anti-CD47 antibody (or immunologically active fragment thereof). In some embodiments, the anti-CD47 antibody is CC-90002 (also known as INBRX103), 5F9 (also known as Hu5F9-G4 and magrolimab), LQ001, HLX24, TI-061, AO-176, SRF-231, IBI-188, IMC-002, SHR-1603, STI-6643, ZL-1201, or an immunologically active fragment of any one of the preceding. In some embodiments, the KRAS G12C inhibitor is a small molecule inhibitor. In some embodiments, the KRAS G12C inhibitor is AMG 510, the structure of which is shown above. In some embodiments, the KRAS G12C inhibitor is MRTX849, the structure of which is shown above.

In some embodiments, provided is a method of treating a KRAS G12C mutant cancer (i.e., a cancer comprising one or more cancer cells that express a KRAS G12C mutant protein) in a subject (e.g., a human subject), comprising administering to the subject an effective amount of an anti-CD47 antibody (or immunologically active fragment thereof) comprising (a) V_{H} domain that comprises (1) a CDR-H1 comprising RAWMN (SEQ ID NO: 5); (2) a CDR-H2 comprising RIKRKTDGETTDYAAPVKG (SEQ ID NO: 6); (3) a CDR-H3 comprising SNRAFDI (SEQ ID NO: 7) and (b) a V_{L} domain that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10) and an effective amount of a small molecule KRAS G12C inhibitor. In some embodiments, provided is a method of stimulating the phagocytosis of a population of cancer cells that comprises one or more cancer cells that express a KRAS G12C mutant protein, comprising contacting the population with an effective amount of an anti-CD47 antibody (or immunologically active fragment thereof) comprising (a) V_{H} domain that comprises (1) a CDR-H1 comprising RAWMN (SEQ ID NO: 5); (2) a CDR-H2 comprising RIKRKTDGETTDYAAPVKG (SEQ ID NO: 6); (3) a CDR-H3 comprising SNRAFDI (SEQ ID NO: 7) and (b) a V_{L} domain that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10) and an effective amount of a small molecule KRAS G12C inhibitor. In some embodiments, the KRAS G12C mutant cancer (or the one or more cancer cells that expresses KRAS G12C mutant protein) is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12C mutant cancer (or the one or more cancer cells that expresses KRAS G12C mutant protein) is NSCLC. In some embodiments of the methods of treatment, the anti-CD47 antibody (or immunologically active fragment thereof) and the small molecule KRAS G12C inhibitor are administered simultaneously. In some embodiments of the methods of treatment, the anti-CD47 antibody (or immunologically active fragment thereof) and the small molecule KRAS G12C inhibitor are administered sequentially. In some embodiments of the methods of treatment, the anti-CD47 antibody (or immunologically active fragment thereof) is administered prior to the small molecule KRAS G12C inhibitor. In some embodiments of the methods of treatment, the small molecule KRAS G12C inhibitor is administered prior to the anti-CD47 antibody (or immunologically active fragment thereof). In some embodiments, the KRAS G12C inhibitor is AMG 510, the structure of which is shown above. In some embodiments, the KRAS G12C inhibitor is MRTX849, the structure of which is shown above.

In some embodiments, provided is a method of treating a KRAS G12C mutant cancer (i.e., a cancer comprising one or more cancer cells that express a KRAS G12C mutant protein) in a subject (e.g., a human subject), comprising administering to the subject an effective amount of an anti-CD47 antibody (or immunologically active fragment thereof) comprising (a) a VH domain that comprises (1) a CDR-H1 comprising RAWMN (SEQ ID NO: 5); (2) a CDR-H2 comprising RIKRKTDGETTDYAAPVKG (SEQ ID NO: 6); (3) a CDR-H3 comprising SNRAFDI (SEQ ID NO: 7) and (b) a VL domain that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10) and an effective amount of a small molecule KRAS G12C inhibitor. In some embodiments, the KRAS G12C mutant cancer or KRAS G12D mutant cancer) is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS mutant cancer is a KRAS G12C mutant cancer. In some embodiments, the KRAS G12C mutant cancer is NSCLC. In some embodiments, the KRAS mutant cancer is a KRAS G12D mutant cancer. In some embodiments, the KRAS G12D mutant cancer is colorectal cancer, e.g., colorectal adenocarcinoma, or colon cancer, e.g., colon adenocarcinoma. In some embodiments of the methods of treatment, the anti-CD47 antibody (or immunologically active fragment thereof) and the small molecule KRAS inhibitor (e.g., the KRAS G12C inhibitor or the KRAS G12D inhibitor) are administered simultaneously. In some embodiments of the methods of treatment, the anti-CD47 antibody (or immunologically active fragment thereof) and the small molecule KRAS inhibitor (e.g., the KRAS G12C inhibitor or the KRAS G12D inhibitor) are administered sequentially. In some embodiments of the methods of treatment, the anti-CD47 antibody (or immunologically active fragment thereof) is administered prior to the small molecule KRAS inhibitor (e.g., the KRAS G12C inhibitor or the KRAS G12D inhibitor). In some embodiments of the methods of treatment, the small molecule KRAS inhibitor (e.g., the KRAS G12C inhibitor or the KRAS G12D inhibitor) is administered prior to the anti-CD47 antibody (or immunologically active fragment thereof). In some embodiments, the KRAS inhibitor is a KRAS G12C inhibitor. In some embodiments, the KRAS G12C inhibitor is AMG 510, the structure of which is shown above. In some embodiments, the KRAS G12C inhibitor is MRTX849, the structure of which is shown above. In some embodiments, the KRAS inhibitor is a KRAS G12D inhibitor. In some embodiments, the KRAS G12D inhibitor is MTRX1133, the structure of which is shown above.

In some embodiments, provided is a method of treating a KRAS mutant cancer (i.e., a cancer comprising one or more cancer cells that express a KRAS G12C mutant protein, e.g., a KRAS G12C mutant protein or a KRAS G12D mutant protein) in a subject (e.g., a human subject), comprising administering to the subject an effective amount of an anti-CD47 antibody (or immunologically active fragment thereof) comprising (a) V_{H} domain that comprises (1) a glutamic acid residue (E) at its N-terminus; (2) a CDR-H1 comprising RAWMN (SEQ ID NO: 5); (3) a CDR-H2 comprising RIKRKTDGETTDYAAPVKG (SEQ ID NO: 6); (4) a CDR-H3 comprising SNRAFDI (SEQ ID NO: 7); and (5) a serine (S) at its C-terminus; and (b) a V_{L} domain that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10) and an effective amount of a small molecule KRAS inhibitor (e.g., a KRAS G12C inhibitor wherein the cancer is a KRAS G12C mutant cancer, or a KRAS G12D inhibitor wherein the cancer is a KRAS G12C mutant cancer). In some embodiments, provided is a method of stimulating the phagocytosis of a population of cancer cells that comprises one or more cancer cells that express a KRAS mutant protein (e.g., a KRAS G12C mutant protein or a KRAS G12D mutant protein), comprising contacting the population with an effective amount of an anti-CD47 antibody (or immunologically active fragment thereof) comprising (a) V_{H} domain that comprises (1) a glutamic acid residue (E) at its N-terminus; (2) a CDR-H1 comprising RAWMN (SEQ ID NO: 5); (3) a CDR-H2 comprising RIKRKTDGETTDYAAPVKG (SEQ ID NO: 6); (4) a CDR-H3 comprising SNRAFDI (SEQ ID NO: 7); and (5) a serine (S) at its C-terminus; and (b) a V_{L} domain that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10) and an effective amount of a small molecule inhibitor (e.g., a KRAS G12C inhibitor wherein the mutant protein is a KRAS G12C mutant protein, or a KRAS G12D inhibitor wherein the mutant protein is a KRAS G12C mutant protein). In some embodiments, the KRAS mutant cancer (e.g., the KRAS G12C mutant cancer or the KRAS G12D mutant cancer) or the one or more cancer cells that expresses KRAS mutant protein (e.g., a KRAS G12C mutant protein or the KRAS G12D mutant protein) is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS mutant cancer is a KRAS G12C mutant cancer. In some embodiments, the population of cancer cells comprises one or more cells that express the KRAS G12C mutant protein. In some embodiments, the KRAS G12C mutant cancer (or the one or more cancer cells that expresses KRAS G12C mutant protein) is NSCLC. In some embodiments, the KRAS mutant cancer is a KRAS G12D mutant cancer. In some embodiments, the population of cancer cells comprises one or more cells that express the KRAS G12D mutant protein. In some embodiments, the KRAS G12D mutant cancer (or the one or more cancer cells that expresses KRAS G12D mutant protein) is colorectal cancer, e.g., colorectal adenocarcinoma, or colon cancer, e.g., colon adenocarcinoma. In some embodiments of the methods of treatment, the anti-CD47 antibody (or immunologically active fragment thereof) and the small molecule KRAS inhibitor (e.g., the KRAS G12C inhibitor or the KRAS G12D inhibitor) are administered simultaneously. In some embodiments of the methods of treatment, the anti-CD47 antibody (or immunologically active fragment thereof) and the small molecule KRAS inhibitor (e.g., the KRAS G12C inhibitor or the KRAS G12D inhibitor) are administered sequentially. In some embodiments of the methods of treatment, the anti-CD47 antibody (or immunologically active fragment thereof) is administered prior to the small molecule KRAS inhibitor (e.g., the KRAS G12C inhibitor or the KRAS G12D inhibitor). In some embodiments of the methods of treatment, the small molecule KRAS inhibitor (e.g., the KRAS G12C inhibitor or the KRAS G12D inhibitor) is administered prior to the anti-CD47 antibody (or immunologically active fragment thereof). In some embodiments, the KRAS inhibitor is a KRAS G12C inhibitor. In some embodiments, the KRAS G12C inhibitor is a small molecule inhibitor. In some embodiments, the KRAS G12C inhibitor is AMG 510, the structure of which is shown above. In some embodiments, the KRAS G12C inhibitor is MRTX849, the structure of which is shown above. In some embodiments, the KRAS inhibitor is a KRAS G12D inhibitor. In some embodiments, the KRAS G12D inhibitor is a small molecule inhibitor. In some embodiments, the KRAS G12D inhibitor is MTRX1133, the structure of which is shown above.

In some embodiments, provided is a method of treating a KRAS G12C mutant cancer (i.e., a cancer comprising one or more cancer cells that express a KRAS G12C mutant protein) in a subject (e.g., a human subject), comprising administering to the subject an effective amount of an anti-CD47 antibody (or immunologically active fragment thereof) comprising (a) V_{H} domain that comprises (1) a glutamic acid residue (E) at its N-terminus; (2) a CDR-H1 comprising RAWMN (SEQ ID NO: 5); (3) a CDR-H2 comprising RIKRKTDGETTDYAAPVKG (SEQ ID NO: 6); (4) a CDR-H3 comprising SNRAFDI (SEQ ID NO: 7); and (5) a serine (S) at its C-terminus; and (b) a V_{L} domain that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10) and an effective amount of a small molecule KRAS G12C inhibitor. In some embodiments, the KRAS G12C mutant cancer is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12C mutant cancer is NSCLC. In some embodiments of the methods of treatment, the anti-CD47 antibody (or immunologically active fragment thereof) and the small molecule KRAS G12C inhibitor are administered simultaneously. In some embodiments of the methods of treatment, the anti-CD47 antibody (or immunologically active fragment thereof) and the small molecule KRAS G12C inhibitor are administered sequentially. In some embodiments of the methods of treatment, the anti-CD47 antibody (or immunologically active fragment thereof) is administered prior to the small molecule KRAS G12C inhibitor. In some embodiments of the methods of treatment, the small molecule KRAS G12C inhibitor is administered prior to the anti-CD47 antibody (or immunologically active fragment thereof). In some embodiments, the KRAS G12C inhibitor is AMG 510, the structure of which is shown above. In some embodiments, the KRAS G12C inhibitor is MRTX849, the structure of which is shown above.

In some embodiments, provided is a method of treating a KRAS G12C mutant cancer (i.e., a cancer comprising one or more cancer cells that express a KRAS G12C mutant protein) in a subject (e.g., a human subject), comprising administering to the subject an effective amount of an anti-CD47 antibody (or immunologically active fragment thereof) comprising (a) V_{H} domain that comprises SEQ ID NO: 1 and (b) a V_{L} domain that comprises SEQ ID NO: 2 and an effective amount of a small molecule KRAS G12C inhibitor. In some embodiments, provided is a method of stimulating the phagocytosis of a population of cancer cells that comprises one or more cancer cells that express a KRAS G12C mutant protein, comprising contacting the population with an effective amount of an anti-CD47 antibody (or immunologically active fragment thereof) comprising (a) V_{H} domain that comprises SEQ ID NO: 1 and (b) a V_{L} domain that comprises SEQ ID NO: 2 and an effective amount of a small molecule KRAS G12C inhibitor. In some embodiments, the KRAS G12C mutant cancer (or the one or more cancer cells that expresses KRAS G12C mutant protein) is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12C mutant cancer (or the one or more cancer cells that expresses KRAS G12C mutant protein) is NSCLC. In some embodiments of the methods of treatment, the anti-CD47 antibody (or immunologically active fragment thereof) and the small molecule KRAS G12C inhibitor are administered simultaneously. In some embodiments of the methods of treatment, the anti-CD47 antibody (or immunologically active fragment thereof) and the small molecule KRAS G12C inhibitor are administered sequentially. In some embodiments of the methods of treatment, the anti-CD47 antibody (or immunologically active fragment thereof) is administered prior to the small molecule KRAS G12C inhibitor. In some embodiments of the methods of treatment, the small molecule KRAS G12C inhibitor is administered prior to the anti-CD47 antibody (or immunologically active fragment thereof). In some embodiments, the KRAS G12C inhibitor is AMG 510, the structure of which is shown above. In some embodiments, the KRAS G12C inhibitor is MRTX849, the structure of which is shown above.

In some embodiments, provided is a method of treating a KRAS G12C mutant cancer (i.e., a cancer comprising one or more cancer cells that express a KRAS G12C mutant protein) in a subject (e.g., a human subject), comprising administering to the subject an effective amount of an anti-CD47 antibody (or immunologically active fragment thereof) comprising (a) V_{H} domain that comprises SEQ ID NO: 1 and (b) a V_{L} domain that comprises SEQ ID NO: 2 and an effective amount of a small molecule KRAS G12C inhibitor. In some embodiments, the KRAS G12C mutant cancer is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12C mutant cancer is NSCLC. In some embodiments of the methods of treatment, the anti-CD47 antibody (or immunologically active fragment thereof) and the small molecule KRAS G12C inhibitor are administered simultaneously. In some embodiments of the methods of treatment, the anti-CD47 antibody (or immunologically active fragment thereof) and the small molecule KRAS G12C inhibitor are administered sequentially. In some embodiments of the methods of treatment, the anti-CD47 antibody (or immunologically active fragment thereof) is administered prior to the small molecule KRAS G12C inhibitor. In some embodiments of the methods of treatment, the small molecule KRAS G12C inhibitor is administered prior to the anti-CD47 antibody (or immunologically active fragment thereof). In some embodiments, the KRAS G12C inhibitor is AMG 510, the structure of which is shown above. In some embodiments, the KRAS G12C inhibitor is MRTX849, the structure of which is shown above.

In some embodiments, provided is a method of treating a KRAS G12D mutant cancer (i.e., a cancer comprising one or more cancer cells that express a KRAS G12D mutant protein) in a subject (e.g., a human subject), comprising administering to the subject an effective amount of an anti-CD47 antibody (or immunologically active fragment thereof) comprising (a) V_{H} domain that comprises (1) a glutamic acid residue (E) at its N-terminus; (2) a CDR-H1 comprising RAWMN (SEQ ID NO: 5); (3) a CDR-H2 comprising RIKRKTDGETTDYAAPVKG (SEQ ID NO: 6); (4) a CDR-H3 comprising SNRAFDI (SEQ ID NO: 7); and (5) a serine (S) at its C-terminus; and (b) a V_{L} domain that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10) and an effective amount of a small molecule KRAS G12D inhibitor. In some embodiments, the KRAS G12D mutant cancer is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12D mutant cancer is colorectal cancer, e.g., colorectal adenocarcinoma, or colon cancer, e.g., colon adenocarcinoma. In some embodiments of the methods of treatment, the anti-CD47 antibody (or immunologically active fragment thereof) and the small molecule KRAS G12D inhibitor are administered simultaneously. In some embodiments of the methods of treatment, the anti-CD47 antibody (or immunologically active fragment thereof) and the small molecule KRAS G12D inhibitor are administered sequentially. In some embodiments of the methods of treatment, the anti-CD47 antibody (or immunologically active fragment thereof) is administered prior to the small molecule KRAS G12D inhibitor. In some embodiments of the methods of treatment, the small molecule KRAS G12D inhibitor is administered prior to the anti-CD47 antibody (or immunologically active fragment thereof). In some embodiments, the KRAS G12D inhibitor is MRTX1122, the structure of which is shown above.

In some embodiments, provided is a method of treating a KRAS G12D mutant cancer (i.e., a cancer comprising one or more cancer cells that express a KRAS G12D mutant protein) in a subject (e.g., a human subject), comprising administering to the subject an effective amount of an anti-CD47 antibody (or immunologically active fragment thereof) comprising (a) V_{H} domain that comprises SEQ ID NO: 1 and (b) a V_{L} domain that comprises SEQ ID NO: 2 and an effective amount of a small molecule KRAS G12D inhibitor. In some embodiments, provided is a method of stimulating the phagocytosis of a population of cancer cells that comprises one or more cancer cells that express a KRAS G12D mutant protein, comprising contacting the population with an effective amount of an anti-CD47 antibody (or immunologically active fragment thereof) comprising (a) V_{H} domain that comprises SEQ ID NO: 1 and (b) a V_{L} domain that comprises SEQ ID NO: 2 and an effective amount of a small molecule KRAS G12D inhibitor. In some embodiments, the KRAS G12D mutant cancer (or the one or more cancer cells that expresses KRAS G12D mutant protein) is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12D mutant cancer (or the one or more cancer cells that expresses KRAS G12D mutant protein) is colorectal cancer, e.g., colorectal adenocarcinoma, or colon cancer, e.g., colon adenocarcinoma. In some embodiments of the methods of treatment, the anti-CD47 antibody (or immunologically active fragment thereof) and the small molecule KRAS G12D inhibitor are administered simultaneously. In some embodiments of the methods of treatment, the anti-CD47 antibody (or immunologically active fragment thereof) and the small molecule KRAS G12D inhibitor are administered sequentially. In some embodiments of the methods of treatment, the anti-CD47 antibody (or immunologically active fragment thereof) is administered prior to the small molecule KRAS G12D inhibitor. In some embodiments of the methods of treatment, the small molecule KRAS G12D inhibitor is administered prior to the anti-CD47 antibody (or immunologically active fragment thereof). In some embodiments, the KRAS G12D inhibitor is MRTX1133, the structure of which is shown above.

In some embodiments, provided is a method of treating a KRAS G12D mutant cancer (i.e., a cancer comprising one or more cancer cells that express a KRAS G12D mutant protein) in a subject (e.g., a human subject), comprising administering to the subject an effective amount of an anti-CD47 antibody (or immunologically active fragment thereof) comprising (a) V_{H} domain that comprises SEQ ID NO: 1 and (b) a V_{L} domain that comprises SEQ ID NO: 2 and an effective amount of a small molecule KRAS G12D inhibitor. In some embodiments, the KRAS G12D mutant cancer is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12D mutant cancer is colorectal cancer, e.g., colorectal adenocarcinoma, or colon cancer, e.g., colon adenocarcinoma. In some embodiments of the methods of treatment, the anti-CD47 antibody (or immunologically active fragment thereof) and the small molecule KRAS G12D inhibitor are administered simultaneously. In some embodiments of the methods of treatment, the anti-CD47 antibody (or immunologically active fragment thereof) and the small molecule KRAS G12D inhibitor are administered sequentially. In some embodiments of the methods of treatment, the anti-CD47 antibody (or immunologically active fragment thereof) is administered prior to the small molecule KRAS G12D inhibitor. In some embodiments of the methods of treatment, the small molecule KRAS G12D inhibitor is administered prior to the anti-CD47 antibody (or immunologically active fragment thereof). In some embodiments, the KRAS G12D inhibitor is MRTX1122, the structure of which is shown above.

In some embodiments, provided is the use of an agent (e.g., therapeutic agent) that blocks the interaction between CD47 (e.g., hCD47) and SIRPα (e.g., hSIRPα) in the manufacture of a medicament for treating a KRAS G12C mutant cancer (i.e., a cancer comprising one or more cancer cells that express a KRAS G12C mutant protein) in a subject (e.g., a human subject), wherein the medicament is for administration with a KRAS G12C inhibitor. In some embodiments, provided is the use of a KRAS G12C inhibitor in the manufacture of a medicament for treating a KRAS G12C mutant cancer (i.e., a cancer comprising one or more cancer cells that express a KRAS G12C mutant protein) in a subject (e.g., a human subject), wherein the medicament is for administration with an agent (e.g., therapeutic agent) that blocks the interaction between CD47 (e.g., hCD47) and SIRPα (e.g., hSIRPα). In some embodiments, provided is the use of an agent (e.g., therapeutic agent) that blocks the interaction between CD47 (e.g., hCD47) and SIRPα (e.g., hSIRPα) in the manufacture of a medicament for stimulating the phagocytosis of a population of cancer cells that comprises one or more cancer cells that express a KRAS G12C mutant protein in a subject (e.g., a human subject), wherein the medicament is for administration with a KRAS G12C inhibitor. In some embodiments, provided is the use of a KRAS G12C inhibitor in the manufacture of a medicament for stimulating the phagocytosis of a population of cancer cells that comprises one or more cancer cells that express a KRAS G12C mutant protein in a subject (e.g., a human subject), wherein the medicament is for administration with an agent (e.g., therapeutic agent) that blocks the interaction between CD47 (e.g., hCD47) and SIRPα (e.g., hSIRPα). In some embodiments, the KRAS G12C mutant cancer (or the one or more cancer cells that expresses KRAS G12C mutant protein) is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12C mutant cancer (or the one or more cancer cells that expresses KRAS G12C mutant protein) is NSCLC. In some embodiments, the agent that blocks the interaction between CD47 and SIRPα is a polypeptide. In some embodiments, the agent that blocks the interaction between CD47 and SIRPα is an antibody, antibody construct, or an immunologically active fragment of the antibody of the antibody construct. In some embodiments, the agent that blocks the interaction between CD47 and SIRPα is an anti-CD47 antibody (or immunologically active fragment thereof). In some embodiments, the anti-CD47 antibody is CC-90002 (also known as INBRX103), 5F9 (also known as Hu5F9-G4 and magrolimab), LQ001, HLX24, TI-061, AO-176, SRF-231, IBI-188, IMC-002, SHR-1603, STI-6643, ZL-1201, or an immunologically active fragment of any one of the preceding. In some embodiments, the anti-CD47 antibody (or immunologically active fragment thereof) is any anti-CD47 antibody (or immunologically active fragment thereof) described herein. In some embodiments, the KRAS G12C inhibitor is a small molecule inhibitor. In some embodiments, the KRAS G12C inhibitor is AMG 510, the structure of which is shown above. In some embodiments, the KRAS G12C inhibitor is MRTX849, the structure of which is shown above. In some embodiments of the methods of treatment, the anti-CD47 antibody (or immunologically active fragment thereof) and the small molecule KRAS G12C inhibitor are administered simultaneously. In some embodiments of the methods of treatment, the anti-CD47 antibody (or immunologically active fragment thereof) and the small molecule KRAS G12C inhibitor are administered sequentially. In some embodiments of the methods of treatment, the anti-CD47 antibody (or immunologically active fragment thereof) is administered prior to the small molecule KRAS G12C inhibitor. In some embodiments of the methods of treatment, the small molecule KRAS G12C inhibitor is administered prior to the anti-CD47 antibody (or immunologically active fragment thereof).

In some embodiments, provided is the use of an agent (e.g., therapeutic agent) that blocks the interaction between CD47 (e.g., hCD47) and SIRPα (e.g., hSIRPα) in the manufacture of a medicament for treating a KRAS G12D mutant cancer (i.e., a cancer comprising one or more cancer cells that express a KRAS G12D mutant protein) in a subject (e.g., a human subject), wherein the medicament is for administration with a KRAS G12D inhibitor. In some embodiments, provided is the use of a KRAS G12D inhibitor in the manufacture of a medicament for treating a KRAS G12D mutant cancer (i.e., a cancer comprising one or more cancer cells that express a KRAS G12D mutant protein) in a subject (e.g., a human subject), wherein the medicament is for administration with an agent (e.g., therapeutic agent) that blocks the interaction between CD47 (e.g., hCD47) and SIRPα (e.g., hSIRPα). In some embodiments, provided is the use of an agent (e.g., therapeutic agent) that blocks the interaction between CD47 (e.g., hCD47) and SIRPα (e.g., hSIRPα) in the manufacture of a medicament for stimulating the phagocytosis of a population of cancer cells that comprises one or more cancer cells that express a KRAS G12D mutant protein in a subject (e.g., a human subject), wherein the medicament is for administration with a KRAS G12D inhibitor. In some embodiments, provided is the use of a KRAS G12D inhibitor in the manufacture of a medicament for stimulating the phagocytosis of a population of cancer cells that comprises one or more cancer cells that express a KRAS G12D mutant protein in a subject (e.g., a human subject), wherein the medicament is for administration with an agent (e.g., therapeutic agent) that blocks the interaction between CD47 (e.g., hCD47) and SIRPα (e.g., hSIRPα). In some embodiments, the KRAS G12D mutant cancer (or the one or more cancer cells that expresses KRAS G12D mutant protein) is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12D mutant cancer (or the one or more cancer cells that expresses KRAS G12D mutant protein) is colorectal cancer, e.g., colorectal adenocarcinoma, or colon cancer, e.g., colon adenocarcinoma. In some embodiments, the agent that blocks the interaction between CD47 and SIRPα is a polypeptide. In some embodiments, the agent that blocks the interaction between CD47 and SIRPα is an antibody, antibody construct, or an immunologically active fragment of the antibody of the antibody construct. In some embodiments, the agent that blocks the interaction between CD47 and SIRPα is an anti-CD47 antibody (or immunologically active fragment thereof). In some embodiments, the anti-CD47 antibody is CC-90002 (also known as INBRX103), 5F9 (also known as Hu5F9-G4 and magrolimab), LQ001, HLX24, TI-061, AO-176, SRF-231, IBI-188, IMC-002, SHR-1603, STI-6643, ZL-1201, or an immunologically active fragment of any one of the preceding. In some embodiments, the anti-CD47 antibody (or immunologically active fragment thereof) is any anti-CD47 antibody (or immunologically active fragment thereof) described herein. In some embodiments, the KRAS G12D inhibitor is a small molecule inhibitor. In some embodiments, the KRAS G12D inhibitor is MRTX1133, the structure of which is shown above. In some embodiments of the methods of treatment, the anti-CD47 antibody (or immunologically active fragment thereof) and the small molecule KRAS G12D inhibitor are administered simultaneously. In some embodiments of the methods of treatment, the anti-CD47 antibody (or immunologically active fragment thereof) and the small molecule KRAS G12D inhibitor are administered sequentially. In some embodiments of the methods of treatment, the anti-CD47 antibody (or immunologically active fragment thereof) is administered prior to the small molecule KRAS G12D inhibitor. In some embodiments of the methods of treatment, the small molecule KRAS G12D inhibitor is administered prior to the anti-CD47 antibody (or immunologically active fragment thereof).

In some embodiments, provided is the use of an agent (e.g., therapeutic agent) that blocks the interaction between CD47 (e.g., hCD47) and SIRPα (e.g., hSIRPα) in a method of treating a KRAS G12C mutant cancer (i.e., a cancer comprising one or more cancer cells that express a KRAS G12C mutant protein) in a subject (e.g., a human subject), wherein the agent is for use (e.g., administered) with a KRAS G12C inhibitor. In some embodiments, provided is the use of a KRAS G12C inhibitor in a method of treating a KRAS G12C mutant cancer (i.e., a cancer comprising one or more cancer cells that express a KRAS G12C mutant protein) in a subject (e.g., a human subject), wherein the KRAS G12C inhibitor is for use (e.g., administered) with an agent (e.g., therapeutic agent) that blocks the interaction between CD47 (e.g., hCD47) and SIRPα (e.g., hSIRPα). In some embodiments, provided is the use of an agent (e.g., therapeutic agent) that blocks the interaction between CD47 (e.g., hCD47) and SIRPα (e.g., hSIRPα) in a method of stimulating the phagocytosis of a population of cancer cells that comprises one or more cancer cells that express a KRAS G12C mutant protein in a subject (e.g., a human subject), wherein the agent is for use (e.g., administered) with a KRAS G12C inhibitor. In some embodiments, provided is the use of a KRAS G12C inhibitor in a method of stimulating the phagocytosis of a population of cancer cells that comprises one or more cancer cells that express a KRAS G12C mutant protein in a subject (e.g., a human subject), wherein the KRAS G12C inhibitor is for use (e.g. administered) with an agent (e.g., therapeutic agent) that blocks the interaction between CD47 (e.g., hCD47) and SIRPα (e.g., hSIRPα). In some embodiments, the KRAS G12C mutant cancer (or the one or more cancer cells that expresses KRAS G12C mutant protein) is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12C mutant cancer (or the one or more cancer cells that expresses KRAS G12C mutant protein) is NSCLC. In some embodiments, the agent that blocks the interaction between CD47 and SIRPα is an antibody, antibody construct, or an immunologically active fragment of the antibody of the antibody construct. In some embodiments, the agent that blocks the interaction between CD47 and SIRPα is an anti-CD47 antibody (or immunologically active fragment thereof). In some embodiments, the anti-CD47 antibody is CC-90002 (also known as INBRX103), 5F9 (also known as Hu5F9-G4 and magrolimab), LQ001, HLX24, TI-061, AO-176, SRF-231, IBI-188, IMC-002, SHR-1603, STI-6643, ZL-1201, or an immunologically active fragment of any one of the preceding. In some embodiments, the anti-CD47 antibody (or immunologically active fragment thereof) is any anti-CD47 antibody (or immunologically active fragment thereof) described herein. In some embodiments, the KRAS G12C inhibitor is a small molecule inhibitor. In some embodiments, the KRAS G12C inhibitor is AMG 510, the structure of which is shown above. In some embodiments, the KRAS G12C inhibitor is MRTX849, the structure of which is shown above. In some embodiments of the methods of treatment, the anti-CD47 antibody (or immunologically active fragment thereof) and the small molecule KRAS G12C inhibitor are administered simultaneously. In some embodiments of the methods of treatment, the anti-CD47 antibody (or immunologically active fragment thereof) and the small molecule KRAS G12C inhibitor are administered sequentially. In some embodiments of the methods of treatment, the anti-CD47 antibody (or immunologically active fragment thereof) is administered prior to the small molecule KRAS G12C inhibitor. In some embodiments of the methods of treatment, the small molecule KRAS G12C inhibitor is administered prior to the anti-CD47 antibody (or immunologically active fragment thereof).

In some embodiments, provided is the use of an agent (e.g., therapeutic agent) that blocks the interaction between CD47 (e.g., hCD47) and SIRPα (e.g., hSIRPα) in a method of treating a KRAS G12D mutant cancer (i.e., a cancer comprising one or more cancer cells that express a KRAS G12D mutant protein) in a subject (e.g., a human subject), wherein the agent is for use (e.g., administered) with a KRAS G12D inhibitor. In some embodiments, provided is the use of a KRAS G12D inhibitor in a method of treating a KRAS G12D mutant cancer (i.e., a cancer comprising one or more cancer cells that express a KRAS G12D mutant protein) in a subject (e.g., a human subject), wherein the KRAS G12D inhibitor is for use (e.g., administered) with an agent (e.g., therapeutic agent) that blocks the interaction between CD47 (e.g., hCD47) and SIRPα (e.g., hSIRPα). In some embodiments, provided is the use of an agent (e.g., therapeutic agent) that blocks the interaction between CD47 (e.g., hCD47) and SIRPα (e.g., hSIRPα) in a method of stimulating the phagocytosis of a population of cancer cells that comprises one or more cancer cells that express a KRAS G12D mutant protein in a subject (e.g., a human subject), wherein the agent is for use (e.g., administered) with a KRAS G12D inhibitor. In some embodiments, provided is the use of a KRAS G12D inhibitor in a method of stimulating the phagocytosis of a population of cancer cells that comprises one or more cancer cells that express a KRAS G12D mutant protein in a subject (e.g., a human subject), wherein the KRAS G12D inhibitor is for use (e.g. administered) with an agent (e.g., therapeutic agent) that blocks the interaction between CD47 (e.g., hCD47) and SIRPα (e.g., hSIRPα). In some embodiments, the KRAS G12D mutant cancer (or the one or more cancer cells that expresses KRAS G12D mutant protein) is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12D mutant cancer (or the one or more cancer cells that expresses KRAS G12D mutant protein) is colorectal cancer, e.g., colorectal adenocarcinoma, or colon cancer, e.g., colon adenocarcinoma. In some embodiments, the agent that blocks the interaction between CD47 and SIRPα is an antibody, antibody construct, or an immunologically active fragment of the antibody of the antibody construct. In some embodiments, the agent that blocks the interaction between CD47 and SIRPα is an anti-CD47 antibody (or immunologically active fragment thereof). In some embodiments, the anti-CD47 antibody is CC-90002 (also known as INBRX103), 5F9 (also known as Hu5F9-G4 and magrolimab), LQ001, HLX24, TI-061, AO-176, SRF-231, IBI-188, IMC-002, SHR-1603, STI-6643, ZL-1201, or an immunologically active fragment of any one of the preceding. In some embodiments, the anti-CD47 antibody (or immunologically active fragment thereof) is any anti-CD47 antibody (or immunologically active fragment thereof) described herein. In some embodiments, the KRAS G12D inhibitor is a small molecule inhibitor. In some embodiments, the KRAS G12D inhibitor is MRTX1133, the structure of which is shown above. In some embodiments of the methods of treatment, the anti-CD47 antibody (or immunologically active fragment thereof) and the small molecule KRAS G12D inhibitor are administered simultaneously. In some embodiments of the methods of treatment, the anti-CD47 antibody (or immunologically active fragment thereof) and the small molecule KRAS G12D inhibitor are administered sequentially. In some embodiments of the methods of treatment, the anti-CD47 antibody (or immunologically active fragment thereof) is administered prior to the small molecule KRAS G12D inhibitor. In some embodiments of the methods of treatment, the small molecule KRAS G12D inhibitor is administered prior to the anti-CD47 antibody (or immunologically active fragment thereof).

In some embodiments, provided is a composition (e.g., pharmaceutical composition) comprising an agent (e.g., therapeutic agent) that blocks the interaction between CD47 (e.g., hCD47) and SIRPα (e.g., hSIRPα) for use in treating a KRAS G12C mutant cancer (i.e., a cancer comprising one or more cancer cells that express a KRAS G12C mutant protein) in a subject (e.g., a human subject), wherein the composition is for use (e.g., administered) with a KRAS G12C inhibitor. In some embodiments, provided is a composition (e.g., a pharmaceutical composition) comprising a KRAS G12C inhibitor for use in treating a KRAS G12C mutant cancer (i.e., a cancer comprising one or more cancer cells that express a KRAS G12C mutant protein) in a subject (e.g., a human subject), wherein the composition inhibitor is for use (e.g., administered) with an agent (e.g., therapeutic agent) that blocks the interaction between CD47 (e.g., hCD47) and SIRPα (e.g., hSIRPα). In some embodiments, provided is a composition (e.g., pharmaceutical composition) comprising an agent (e.g., therapeutic agent) that blocks the interaction between CD47 (e.g., hCD47) and SIRPα (e.g., hSIRPα) for use in stimulating the phagocytosis of a population of cancer cells that comprises one or more cancer cells that express a KRAS G12C mutant protein in a subject (e.g., a human subject), wherein the composition is for use (e.g., administered) with a KRAS G12C inhibitor. In some embodiments, provided is a composition comprising a KRAS G12C for stimulating the phagocytosis of a population of cancer cells that comprises one or more cancer cells that express a KRAS G12C mutant protein in a subject (e.g., a human subject), wherein the composition is for use (e.g. administered) with an agent (e.g., therapeutic agent) that blocks the interaction between CD47 (e.g., hCD47) and SIRPα (e.g., hSIRPα). In some embodiments, the KRAS G12C mutant cancer (or the one or more cancer cells that expresses KRAS G12C mutant protein) is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12C mutant cancer (or the one or more cancer cells that expresses KRAS G12C mutant protein) is NSCLC. In some embodiments, the agent that blocks the interaction between CD47 and SIRPα is an antibody, antibody construct, or an immunologically active fragment of the antibody of the antibody construct. In some embodiments, the agent that blocks the interaction between CD47 and SIRPα is an anti-CD47 antibody (or immunologically active fragment thereof). In some embodiments, the anti-CD47 antibody is CC-90002 (also known as INBRX103), 5F9 (also known as Hu5F9-G4 and magrolimab), LQ001, HLX24, TI-061, AO-176, SRF-231, IBI-188 (also known as letaplimab), IMC-002, SHR-1603, STI-6643, ZL-1201, or an immunologically active fragment of any one of the preceding. In some embodiments, the anti-CD47 antibody (or immunologically active fragment thereof) is any anti-CD47 antibody (or immunologically active fragment thereof) described herein. In some embodiments, the KRAS G12C inhibitor is a small molecule inhibitor. In some embodiments, the KRAS G12C inhibitor is AMG 510, the structure of which is shown above. In some embodiments, the KRAS G12C inhibitor is MRTX849, the structure of which is shown above. In some embodiments of the methods of treatment, the anti-CD47 antibody (or immunologically active fragment thereof) and the small molecule KRAS G12C inhibitor are administered simultaneously. In some embodiments of the methods of treatment, the anti-CD47 antibody (or immunologically active fragment thereof) and the small molecule KRAS G12C inhibitor are administered sequentially. In some embodiments of the methods of treatment, the anti-CD47 antibody (or immunologically active fragment thereof) is administered prior to the small molecule KRAS G12C inhibitor. In some embodiments of the methods of treatment, the small molecule KRAS G12C inhibitor is administered prior to the anti-CD47 antibody (or immunologically active fragment thereof).

In some embodiments, provided is a composition (e.g., pharmaceutical composition) comprising an agent (e.g., therapeutic agent) that blocks the interaction between CD47 (e.g., hCD47) and SIRPα (e.g., hSIRPα) for use in treating a KRAS G12D mutant cancer (i.e., a cancer comprising one or more cancer cells that express a KRAS G12D mutant protein) in a subject (e.g., a human subject), wherein the composition is for use (e.g., administered) with a KRAS G12D inhibitor. In some embodiments, provided is a composition (e.g., a pharmaceutical composition) comprising a KRAS G12D inhibitor for use in treating a KRAS G12D mutant cancer (i.e., a cancer comprising one or more cancer cells that express a KRAS G12D mutant protein) in a subject (e.g., a human subject), wherein the composition inhibitor is for use (e.g., administered) with an agent (e.g., therapeutic agent) that blocks the interaction between CD47 (e.g., hCD47) and SIRPα (e.g., hSIRPα). In some embodiments, provided is a composition (e.g., pharmaceutical composition) comprising an agent (e.g., therapeutic agent) that blocks the interaction between CD47 (e.g., hCD47) and SIRPα (e.g., hSIRPα) for use in stimulating the phagocytosis of a population of cancer cells that comprises one or more cancer cells that express a KRAS G12D mutant protein in a subject (e.g., a human subject), wherein the composition is for use (e.g., administered) with a KRAS G12D inhibitor. In some embodiments, provided is a composition comprising a KRAS G12D for stimulating the phagocytosis of a population of cancer cells that comprises one or more cancer cells that express a KRAS G12D mutant protein in a subject (e.g., a human subject), wherein the composition is for use (e.g. administered) with an agent (e.g., therapeutic agent) that blocks the interaction between CD47 (e.g., hCD47) and SIRPα (e.g., hSIRPα). In some embodiments, the KRAS G12D mutant cancer (or the one or more cancer cells that expresses KRAS G12D mutant protein) is lung cancer (e.g., lung adenocarcinoma or NSCLC), colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal carcinoma. In some embodiments, the KRAS G12D mutant cancer (or the one or more cancer cells that expresses KRAS G12D mutant protein) is colorectal cancer, e.g., colorectal adenocarcinoma, or colon cancer, e.g., colon adenocarcinoma. In some embodiments, the agent that blocks the interaction between CD47 and SIRPα is an antibody, antibody construct, or an immunologically active fragment of the antibody of the antibody construct. In some embodiments, the agent that blocks the interaction between CD47 and SIRPα is an anti-CD47 antibody (or immunologically active fragment thereof). In some embodiments, the anti-CD47 antibody is CC-90002 (also known as INBRX103), 5F9 (also known as Hu5F9-G4 and magrolimab), LQ001, HLX24, TI-061, AO-176, SRF-231, IBI-188 (also known as letaplimab), IMC-002, SHR-1603, STI-6643, ZL-1201, or an immunologically active fragment of any one of the preceding. In some embodiments, the anti-CD47 antibody (or immunologically active fragment thereof) is any anti-CD47 antibody (or immunologically active fragment thereof) described herein. In some embodiments, the KRAS G12D inhibitor is a small molecule inhibitor. In some embodiments, the KRAS G12D inhibitor is MRTX1133, the structure of which is shown above. In some embodiments of the methods of treatment, the anti-CD47 antibody (or immunologically active fragment thereof) and the small molecule KRAS G12D inhibitor are administered simultaneously. In some embodiments of the methods of treatment, the anti-CD47 antibody (or immunologically active fragment thereof) and the small molecule KRAS G12D inhibitor are administered sequentially. In some embodiments of the methods of treatment, the anti-CD47 antibody (or immunologically active fragment thereof) is administered prior to the small molecule KRAS G12D inhibitor. In some embodiments of the methods of treatment, the small molecule KRAS G12D inhibitor is administered prior to the anti-CD47 antibody (or immunologically active fragment thereof).

### Articles of Manufacture and Kits

Provided is an article of manufacture comprising materials useful for the treatment of a KRAS mutant cancer (e.g., a KRAS G12C mutant cancer or a KRAS G12D mutant cancer). In some embodiments, the KRAS mutant cancer (e.g., a KRAS G12C mutant cancer or a KRAS G12D mutant cancer) is lung adenocarcinoma, non-small cell lung cancer, colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal adenocarcinoma). In certain embodiments, the article of manufacture or kit comprises a container containing a therapeutic agent that blocks the interaction between CD47 (e.g., hCD47) and SIRPα (e.g., hSIRPα). In some embodiments, such therapeutic agent is a polypeptide, e.g., a polypeptide that binds CD47 (e.g., hCD47). In some embodiments, the agent that blocks the interaction between CD47 and SIRPα is an antibody, antibody construct, or an immunologically active fragment of the antibody or antibody construct. In some embodiments, the agent that blocks the interaction between CD47 and SIRPα is an anti-CD47 antibody or immunologically active fragment thereof. In some embodiments, the anti-CD47 antibody (or immunologically active fragment thereof) is CC-90002 (also known as INBRX103), 5F9 (also known as Hu5F9-G4 and magrolimab), LQ001, HLX24, TI-061, AO-176, SRF-231, IBI-188, IMC-002, SHR-1603, STI-6643, or ZL-1201. In some embodiments, the anti-CD47 antibody or immunologically active fragment thereof is an anti-CD47 antibody (or fragment thereof) described herein or a pharmaceutical composition comprising such an anti-CD47 antibody or antibody fragment. In certain embodiments, the article of manufacture or kit comprises a container containing nucleic acid(s) encoding an anti-CD47 antibody (or an immunologically active fragment thereof), e.g., an anti-CD47 antibody (or fragment) described herein. In some embodiments, the kit includes a cell of cell line that produces an anti-CD47 antibody (or immunologically active fragment thereof) described herein. In some embodiments, the kit includes one or more positive controls, for example CD47 (or fragments thereof) or CD47+ cells. In some embodiments, the kit includes negative controls, for example a surface or solution that is substantially free of CD47, or a cell that does not express CD47.

In certain embodiments, the article of manufacture or kit comprises a container and a label or package insert on or associated with the container. Suitable containers include, for example, bottles, vials, syringes, IV solution bags, test tubes, etc. The containers may be formed from a variety of materials such as glass or plastic. The container holds a composition (e.g., a composition comprising an anti-CD47 antibody or immunologically active fragment thereof) which is by itself or combined with another composition effective for treating (such as delaying the progression of) a KRAS mutant cancer (e.g., a KRAS G12C mutant cancer or a KRAS G12D mutant cancer) such as lung adenocarcinoma, non-small cell lung cancer, colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal adenocarcinoma). The container may have a sterile access port (for example the container may be an intravenous solution bag or a vial having a stopper pierceable by a hypodermic injection needle). At least one agent in the composition is an agent that blocks the binding of hCD47 to hSIRPα. In some embodiments, the at least one agent in the composition is an anti-CD47 antibody (or immunologically active fragment thereof), e.g., an anti-CD47 antibody described herein. In some embodiments, the label or package insert indicates that the composition is used in combination with a KRAS G12C inhibitor for treating a KRAS G12C mutant cancer (such as lung adenocarcinoma, non-small cell lung cancer, colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal adenocarcinoma). In some embodiments, the KRAS G12C mutant cancer is lung cancer, e.g., lung adenocarcinoma, or NSCLC. In some embodiments, the label or package insert indicates that the composition is used in combination with a KRAS G12D inhibitor for treating a KRAS G12D mutant cancer (such as lung adenocarcinoma, non-small cell lung cancer, colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal adenocarcinoma). In some embodiments, the KRAS G12D mutant cancer is colorectal cancer, e.g., colorectal adenocarcinoma, or colon cancer, e.g., colon adenocarcinoma.

In some embodiments, the article of manufacture or kit is for the treatment of a KRAS G12C cancer (e.g., a KRAS G12C cancer described herein) and comprises (a) a first container with a composition contained therein, wherein the composition comprises an is an agent that blocks the binding of hCD47 to hSIRPα (such as an anti-CD47 antibody, or immunologically active fragment thereof, e.g., an anti-CD47 antibody described herein or a fragment thereof), and (b) a second container with a composition contained therein, wherein the composition comprises KRAS G12C inhibitor (e.g., a polypeptide, antibody, fusion polypeptide, antisense oligonucleotide or a small molecule drug that is capable of inhibiting the activity of a KRAS G12C mutant protein). In some embodiments, the second container contains a small molecule KRAS G12C inhibitor. Exemplary small molecule KRAS G12C inhibitors that can be packaged with the kits provided herein include, without limitation, e.g., AMG 510 (Amgen), MRTX849 (Mirati), JAB-21822 (Jacobiopharma), GDC-6036 (Genentech), JDQ443 (Novartis), D-1553 (InventisBio and Merck Sharp & Dohme), GH35 (Genhouse Bio), GFH925 (GenFleet Therapeutics), BPI-421286 (Bettapharma), and LY3537982. In some embodiments, the article of manufacture or kit is for the treatment of a KRAS G12D cancer (e.g., a KRAS G12D cancer described herein) and comprises (a) a first container with a composition contained therein, wherein the composition comprises an is an agent that blocks the binding of hCD47 to hSIRPα (such as an anti-CD47 antibody, or immunologically active fragment thereof, e.g., an anti-CD47 antibody described herein or a fragment thereof), and (b) a second container with a composition contained therein, wherein the composition comprises KRAS G12D inhibitor (e.g., a polypeptide, antibody, fusion polypeptide, antisense oligonucleotide or a small molecule drug that is capable of inhibiting the activity of a KRAS G12D mutant protein). In some embodiments, the second container contains a small molecule KRAS G12D inhibitor. Exemplary small molecule KRAS G12D inhibitors that can be packaged with the kits provided herein include, without limitation, e.g., MRTX1133 (Mirati). Additionally, the article of manufacture may further comprise an additional container comprising a pharmaceutically acceptable buffer, such as bacteriostatic water for injection (BWFI), phosphate-buffered saline, Ringer's solution, and dextrose solution. It may further include other materials desirable from a commercial and user standpoint, including other buffers, diluents, filters, needles, and syringes.

All publications and patent applications cited in this specification are herein incorporated by reference as if each individual publication or patent application were specifically and individually indicated to be incorporated by reference.

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. In Vivo Anti-Tumor Efficacy of an Anti-CD47 Antibody in Combination with a KRAS G12C Inhibitor in Mice Bearing Human Non-Small Cell Lung Cancer (NSCLC) Tumor Xenografts

The anti-tumor efficacy of anti-CD47 antibody TJC4 in combination with the KRAS G12C inhibitor AMG-510 was assessed in mice bearing human NCI-H358 non-small cell lung cancer (NSCLC) tumor xenografts. The NCI-H358 cell line, which was established from a chemotherapy-naïve NSCLC tumor, harbors a KRAS G12C mutation. TJC4 is an exemplary anti-CD47 antibody that comprises a V_{H} that comprises SEQ ID NO: 1, a V_{L} that comprises SEQ ID NO: 2, and a human IgG4 Fe region that comprises an S228P substitution (EU numbering). AMG-510 is also known as Sotorasib. KRAS G12C mutations, which are common in NSCLC, lead to constitutive activation of the tumor growth-promoting RAS/MAPK signaling pathway.

Briefly, NCI-H358 cells (ATCC, CRL-5807) were cultured in RPMI-1640 medium supplemented with 10% fetal bovine serum at 37° C. Next, each of 32 CB17 SCID mice (female, age: 6-8 weeks, body weight: 16-20 g) were subcutaneously inoculated to the right back with 5×10⁶ NCI-H358 cells (resuspended in 0.2 mL DPBS comprising 50% Matrigel. Seven days later, when tumor volume reached 70-80 mm³, animals were randomly divided into four groups (n=8/group) and administered with (a) PBS (control), (b) AMG-510 monotherapy, (c) TJC4 monotherapy, or (d) AMG-510 in combination TJC4, according to the dosages and administration frequencies shown in Table 1 below.

TABLE 1
Drug Dosages and Administration Schedules

| | Dosage | Administration | Administration |
|---|---|---|---|
| Group | (mg/kg) | Route | Frequency |
| (a) PBS | N/A | i.p. | QD |
| (b) AMG-510 | 1 | p.o. | QD |
| (c) TJC4 | 1 | i.p. | QD |
| (d) AMG-510 + TJC4 | 1 + 1 | p.o. + i.p. | QD + QD |

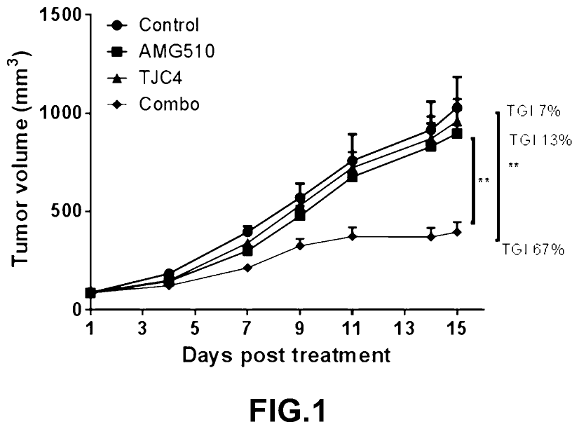

Each animal's tumor size was measured three times per week for 15 days. Tumor size was calculated as V=0.5L×W², where L and W indicate long and short diameters of the tumor, respectively. Mean tumor size of animals in each group was analyzed, as shown in FIG. 1. Anti-tumor efficacy of the drugs was also analyzed by TGI % (percentage of tumor growth inhibition). TGI % was calculated as:

$$TGI ( \% ) = [ 1 - ( {V}_{N} - {V}_{0} ) / ( {V}_{CN} - {V}_{C 0} ) ] * 100 \% ,$$

where V_{N} is mean tumor volume on day N of administration, V₀ is mean tumor volume on day 0 of administration, V_{CN} is mean tumor volume in control group on day N of administration, and V_{C0} is mean tumor volume in control group on day 0 administration.

T/C (relative tumor inhibition) was calculated as:

$$T / C \% = TRTV / CRTV \times 100 \% ,$$

where TRTV is treatment group RTV, and CRTV is control group RTV. RTV=V_{N}/V₁, where V₁ is the mean tumor volume measured on Day 1 of administration, and V_{N} is the mean tumor volume measured on Day N of administration.

As shown in FIG. 1, mice treated with either single agent anti-CD47 antibody TJC4 or single agent KRAS G12C inhibitor AMG-510 did not demonstrate tumor growth inhibition as compared to mice treated with PBS control. By contrast, significant tumor growth inhibition was observed in mice treated with TJC4 in combination AMG-510.

The synergy of the combination of TJC4 and AMG-510 was calculated as:

$$Q = ( {TGI}_{AMG - 510 + TJC 4} / ( {TGI}_{AMG - 510} + {TGI}_{TJC 4 -} {TGI}_{AMG - 510} * {TGI}_{TJC 4} ) ,$$

where Q≥1.15 indicates synergistic effect between TJC4 and AMG-510, 0.85≤Q<1 indicates additive effect between TJC4 and AMG-510, and Q<0.85 indicates antagonistic effect between TJC4 and AMG-510. On Day 15, the Q value of TJC4 and AMG-510 was 3.33, indicating that anti-tumor effect of the drug combination is synergistic.

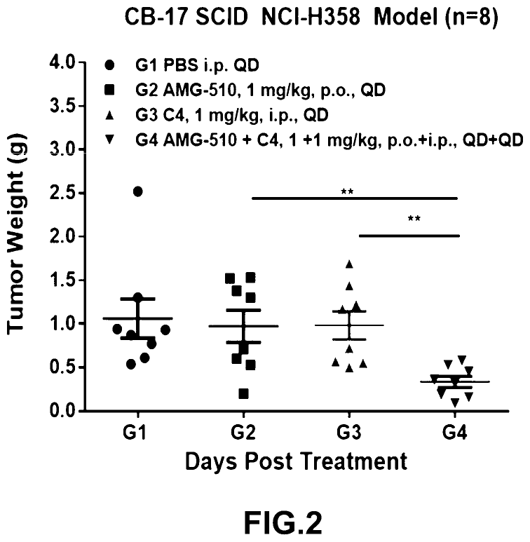

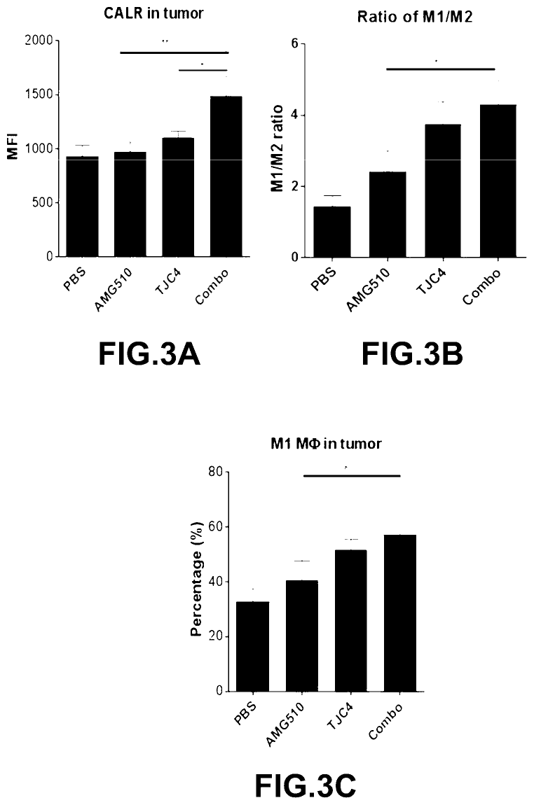

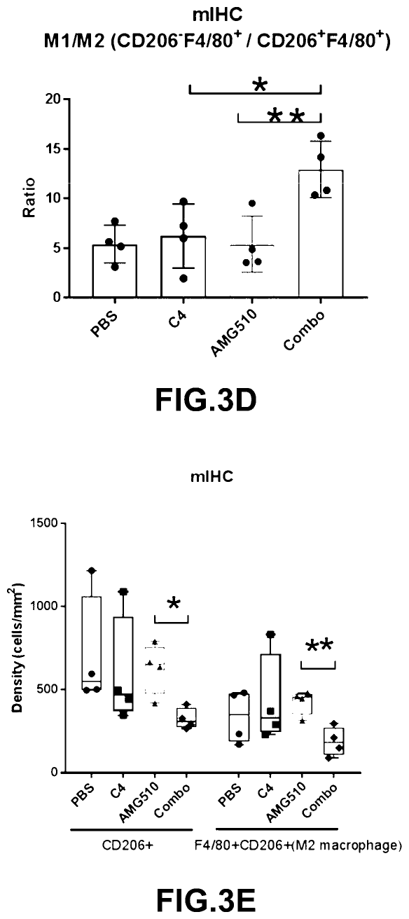

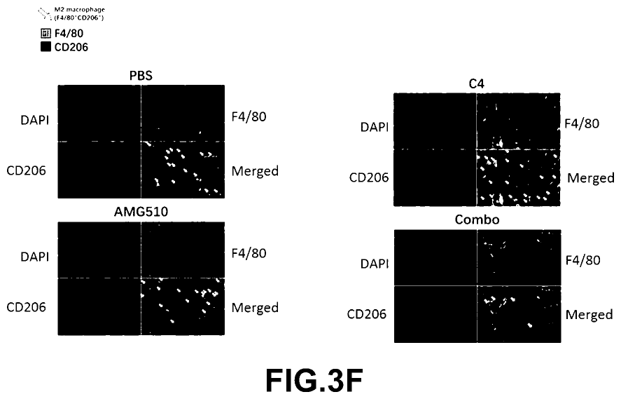

On Day 15, the mice were sacrificed, and their tumor tissues were isolated, weighed, and dispersed into single cell suspensions. The cells in each suspension were subject to immunophenotyping analysis by flow cytometry. It was found that the tumor weight of mice treated with TJC4 in combination with AMG-510 was significantly lower than the tumor weight of mice treated with either single agent TJC4 or single agent AMG-510. See FIG. 2. As shown in FIG. 3A, the tumors of mice treated with TJC4 in combination with AMG-510 showed significantly increased levels of calreticulin “CALR” (i.e., a prophagocytic signal) as compared to the tumors of mice treated with either drug as a single agent. As shown in FIGS. 3B and 3C, the M1:M2 ratio and M1 macrophage percentage was significantly increased in the tumor cells of mice treated with TJC4 in combination with AMG-51 as compared to tumor cells in mice given AMG 510 monotherapy

At the end of study, tumor tissue was obtained from the mice, embedded in paraffin, and fixed with formalin. The FFPE (formalin-fixed paraffin embedded) samples were then subject to tumor immune profiling via multiplexed immunohistochemistry. As shown in FIGS. 3D, and 3E, tumors from mice treated with TJC4 and AMG 510 showed an significantly increased ratio of M1:M2 and a significantly decreased percentage of M2 macrophages as compared to tumors of mice treated with either drug as a single agent. Images of M1 and M2 macrophage populations in tumors from each treatment group are shown in FIG. 3F (M2 macrophages are indicated with white arrows). Both M1 and M2 macrophage are F4/80+; only M2 macrophage are CD206+.

### Example 2. Effects of the Combination of an Anti-CD47 Antibody with a KRAS G12C Inhibitor on the Phagocytosis of Human Non-Small Cell Lung Cancer (NSCLC) Cells In Vitro

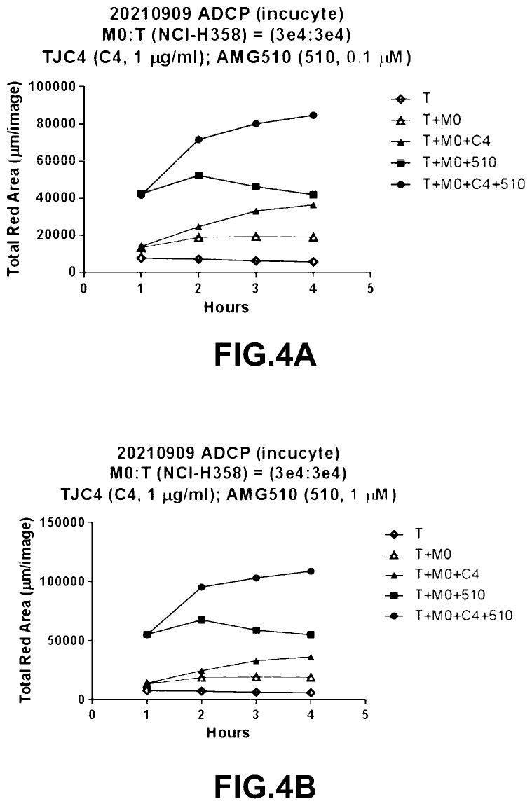

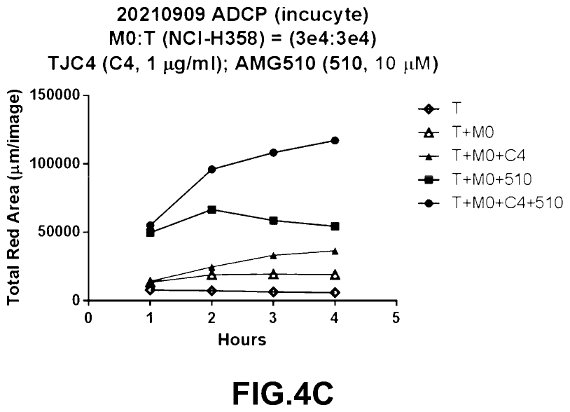

The following experiments were performed to assess the effects of anti-CD47 antibody TJC4 in combination with the KRAS G12C inhibitor AMG-510 on the in vitro phagocytosis of NCI-H358 human NSCLC tumor cells. As discussed in Example 1, NCI-H358 cells bear the KRAS G12C mutation. The in vitro phagocytosis assays were performed by co-culturing M0 macrophage cells with INCUCYTE® PHRODO®-labeled NCI-H358 tumor cells. Briefly, prior to co-culture, NCI-H358 cells were pre-treated with AMG 510 at the concentrations of 0 μm, 0.1 μm, 1 μm, or 10 μm for 24 hours. The NCI-H358 cells were further incubated with M0 macrophage (at a ratio of 1:1) and 1 μg/ml TJC4 for 3-6 hours. The NCI-H358 cells were then subject to INCUCYTE® ZOOM Live-Cell Imaging System analysis, and the percentage of phagocytosed NCI-H358 was calculated. FIG. 4A-4C show that AMG 510 enhanced TJC4-mediated phagocytosis of NCI-H358 by macrophages.

Synergy of the combination of AMG 510 and TJC4 in inducing phagocytosis was calculated as:

$$F = ( Combo / Blank ) / ( ( C 4 / Blank ) * ( SoC / Blank ) ) .$$

An F value≥1.00 indicates a synergistic effect between AMG-510 and TJC4. As shown in Table 2, the combination of anti-CD47 antibody TJC4 and the KRAS G12C inhibitor AMG-510 is synergistic in inducing phagocytosis of tumor cells at all concentrations of AMG-510 tested.

TABLE 2
Combination of the anti-CD47 antibody with KRAS G12C inhibitor
synergistically enhance phagocytosis of tumor cells

| Concentration of AMG-510 | 10 μM | 1 μM | 0.1 μM |
|---|---|---|---|
| F Value | 1.13 | 1.04 | 1.06 |

### Example 3. Effects of the Combination of an Anti-CD47 Antibody with a KRAS G12D Inhibitor on the Phagocytosis of Human Colon Cancer Cells by Macrophages In Vitro

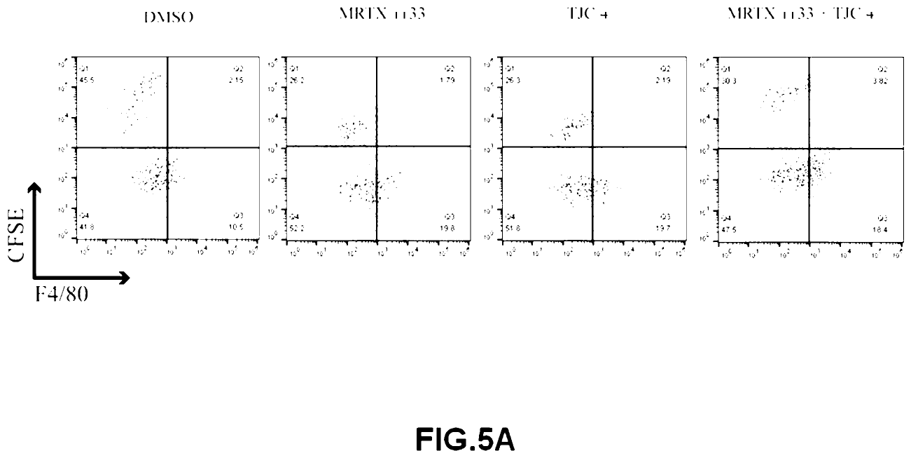

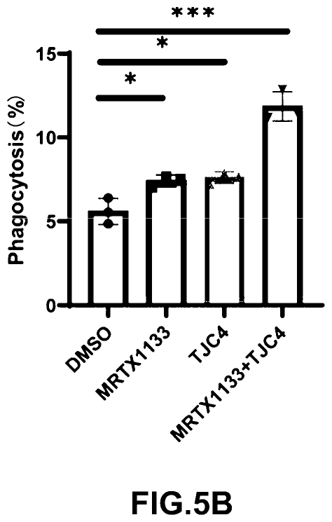

LS180 human colon adenocarcinoma cells, which harbor the KRAS G12D mutation were incubated with (a) DMSO, (b) the KRAS G12D inhibitor MRTX1133 (100 nM), (c) anti-CD47 antibody TJC4 (0.5 μg/ml), or (d) MRTX1133 (100 nM)+TJC4 (0.5 μg/ml) for 24 hours and then labeled with 2.5 μM carboxyfluorescein succinimidyl ester (CFSE) for 10 minutes at 37° C. Macrophage cells, which were obtained by human THP-1 leukemia monocyte cells with lipopolysaccharide (LPS) for 24 hours, were cultured in a serum free medium for 2 hours and then added to 2×10⁵ CFSE-labeled LS180 cells. The macrophages and the LS180 cells were co-cultured at 37° C. for 2 hours. Macrophages were then stained with an APC-labeled anti-F4/80 antibody, and CFSE+ F4/80+ cells were detected by flow cytometry (10,000 cells analyzed in each sample). The percentage of macrophage-phagocytosed LS180 cells was calculated by dividing the number of CFSE+ F4/80+ cells (see the top right quadrant, i.e., Q2, of each flow cytometry histogram in FIG. 5A) by the number CFSE+ cells (see the top left and right quadrants, i.e., Q1 and Q2, respectively) of each flow cytometry histogram in FIG. 5A). In other words, macrophage-phagocytosed LS180 cells (%)=[Q2/(Q1+Q2)]×100%. As shown in FIG. 5B, both single agent MRTX1133 and single agent TJC4 increased the phagocytosis of LS180 cells by macrophages. The combination of MRTX1133 and TJC4 significantly increased the phagocytosis of LS180 cells by macrophages, as compared to either agent alone (see FIG. 5B). The F value was 1.1, suggesting MRTX1133 and TJC4 antibody showed synergistical effect on phagocytosis against G12D mutant colon tumor cells.

### Example 4. Assessing the Effects of an Anti-CD47 Antibody in Combination with a KRAS G12D Inhibitor on Apoptosis of Human Colon Cancer Cells

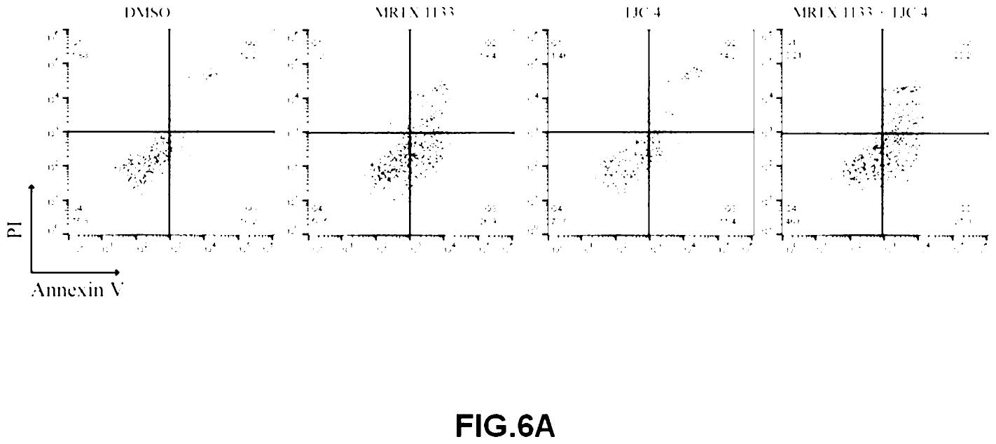

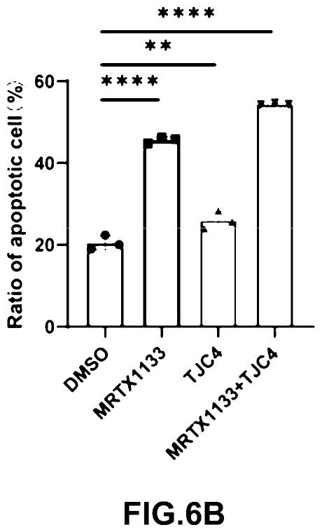

LS180 human colon adenocarcinoma cells were incubated with (a) DMSO, (b) the KRAS G12D inhibitor MRTX1133 (100 nM), (c) anti-CD47 antibody TJC4 (0.5 μg/ml), or (d) MRTX1133 (100 nM)+TJC4 (0.5 μg/ml) for 24 hours and then labeled with fluorescein isothiocyanate (FITC)-Annexin V and propidium iodide (PI). Annexin V (or Annexin A5) is a member of the annexin family of intracellular proteins that binds to phosphatidylserine (PS) in a calcium-dependent manner. PS is normally only found on the intracellular leaflet of the plasma membrane in healthy cells, but during early apoptosis, membrane asymmetry is lost, and PS translocates to the external leaflet. FITC-labeled Annexin V can then be used to specifically target and identify apoptotic cells. PI a cell impermeable nucleic acid intercalating dye. Following staining, the cells were analyzed via flow cytometry. As shown in FIG. 6A, both single agent MRTX1133 and single agent TJC4 increased apoptosis of LS180 cells. The combination of MRTX1133 and TJC4 significantly increased apoptosis LS180 cells, as compared to either agent alone (see FIG. 6B). The F value was 1.03, suggesting MRTX1133 and TJC4 antibody showed synergistical effect on the induction of apoptosis on G12D mutant colon tumor cells.

### Example 5. In Vivo Anti-Tumor Efficacy of an Anti-CD47 Antibody in Combination with a KRAS G12D Inhibitor in Mice Bearing Human Colon Cancer Tumor Xenografts

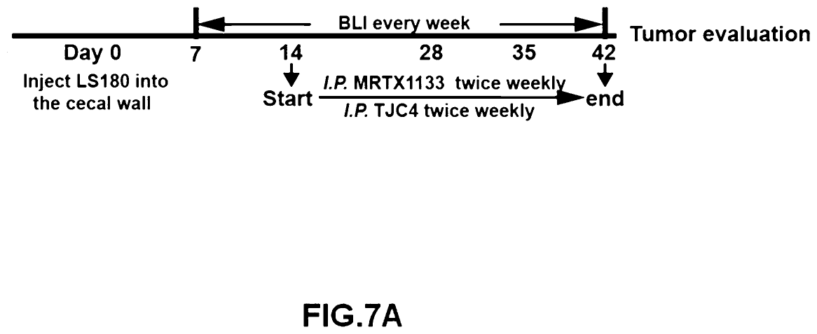

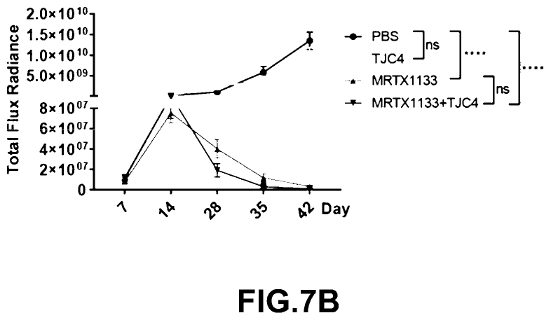

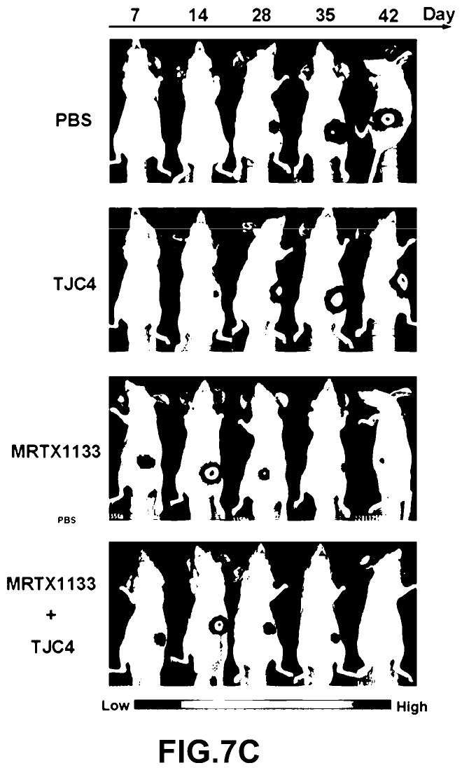

The anti-tumor efficacy of anti-CD47 antibody TJC4 in combination with the KRAS G12D inhibitor MRTZ1133 was assessed in mice bearing human LS180 colon adenocarcinoma tumor xenografts. Briefly, 5×10⁵ LS180 cells were injected into the cecal walls of BALB/c nude mice. Tumors formed and grew stably after 2 weeks. Mice were given one of the following treatments for four weeks: (a) PBS (control); (b) TJC4 (1 mg/kg, intraperitoneally twice a week); (c) MRTX1133 (20 mg/kg, intraperitoneally twice a week); or (d) MRTX1133 (20 mg/kg, intraperitoneally twice a week)+TJC4 (1 mg/kg, intraperitoneally twice a week). See FIG. 7A. Tumor growth was evaluated by Bioluminescence imaging (BLI) every week. As shown in FIG. 7B, single agent MRTX1133 inhibited tumor growth. Single agent TJC4 had a weak inhibitory effect on tumor growth. The combination of MRTX1133 and TJC4 demonstrated increased tumor growth inhibition as compared to either agent alone. Notably, combination treatment led to complete tumor regression. See FIG. 7C. The synergistic factor Q was 2.4492, suggesting MRTX1133 and TJC4 antibody work synergistically to suppress tumor growth.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

| SEQ | | |
|---|---|---|
| ID | | |
| NO. | Sequence | Description |
| 1 | EVQLVESGGGLVKPGGSLRLSCAASGLTFERAWMNWVR | VH of TJC4 |
| | QAPGKGLEWVGRIKRKTDGETTDYAAPVKGRFSISRDDS | |
| | KNTLYLQMNSLKTEDTAVYYCAGSNRAFDIWGQGTMVT | |
| | VSS | |
| 2 | DIVMTQSPDSLAVSLGERATINCKSSQSVLYAGNNRNYL | VL of TJC4 |
| | AWYQQKPGQPPKLLINQASTRASGVPDRFSGSGSGTEFTL | |
| | IISSLQAEDVAIYYCQQYYTPPLAFGGGTKLEIK | |
| 3 | EVQLVESGGGLVKPGGSLRLSCAASGLTFERAWMNWVR | HC of TJC4 |
| | QAPGKGLEWVGRIKRKTDGETTDYAAPVKGRFSISRDDS | |
| | KNTLYLQMNSLKTEDTAVYYCAGSNRAFDIWGQGTMVT | |
| | VSSASTKGPSVFPLAPCSRSTSESTAALGCLVKDYFPEPVT | |
| | VSWNSGALTSGVHTFPAVLQSSGLYSLSSVVTVPSSSLGT | |
| | KTYTCNVDHKPSNTKVDKRVESKYGPPCPPCPAPEFLGG | |
| | PSVFLFPPKPKDTLMISRTPEVTCVVVDVSQEDPEVQFNW | |
| | YVDGVEVHNAKTKPREEQFNSTYRVVSVLTVLHQDWLN | |
| | GKEYKCKVSNKGLPSSIEKTISKAKGQPREPQVYTLPPSQ | |
| | EEMTKNQVSLTCLVKGFYPSDIAVEWESNGQPENNYKTT | |
| | PPVLDSDGSFFLYSRLTVDKSRWQEGNVFSCSVMHEALH | |
| | NHYTQKSLSLSLGK | |
| 4 | DIVMTQSPDSLAVSLGERATINCKSSQSVLYAGNNRNYL | LC of TJC4 |
| | AWYQQKPGQPPKLLINQASTRASGVPDRFSGSGSGTEFTL | |
| | IISSLQAEDVAIYYCQQYYTPPLAFGGGTKLEIKRTVAAPS | |
| | VFIFPPSDEQLKSGTASVVCLLNNFYPREAKVQWKVDNA | |
| | LQSGNSQESVTEQDSKDSTYSLSSTLTLSKADYEKHKVY | |
| | ACEVTHQGLSSPVTKSFNRGEC | |
| 5 | RAWMN | HCDR1 of TJC4 |
| | | (Kabat) |
| 6 | RIKRKTDGETTDYAAPVKG | HCDR2 of TJC4 |
| | | (Kabat) |
| 7 | SNRAFDI | HCDR3 of TJC4 |
| | | (Kabat) |
| 8 | KSSQSVLYAGNNRNYLA | LCDR1 of TJC4 |
| | | (Kabat) |
| 9 | QASTRAS | LCDR2 of TJC4 |
| | | (Kabat) |
| 10 | QQYYTPPLA | LCDR3 of TJC4 |
| | | (Kabat) |
| 11 | GLTFERA | CDR-H1of TJC4 |
| | | (Chothia) |
| 12 | KRKTDGET | CDR-H2 of TJC4 |
| | | (Chothia) |
| 13 | SNRAFDI | CDR-H3 of TJC4 |
| | | (Chothia) |
| 14 | KSSQSVLYAGNNRNYLA | CDR-L1 of TJC4 |
| | | (Chothia) |
| 15 | QASTRAS | CDR-L2 of TJC4 |
| | | (Chothia) |
| 16 | QQYYTPPLA | CDR-L3 of TJC4 |
| | | (Chothia) |
| 17 | GLTFERAW | CDR-H1 of TJC4 |
| | | (IMGT) |
| 18 | IKRKTDGETT | CDR-H2 of TJC4 |
| | | (IMGT) |
| 19 | AGSNRAFDI | CDR-H3 of TJC4 |
| | | (IMGT) |
| 20 | QSVLYAGNNRNY | CDR-L1 of TJC4 |
| | | (IMGT) |
| 21 | QAS | CDR-L2 of TJC4 |
| | | (IMGT) |
| 22 | QQYYTPPLA | CDR-L3 of TJC4 |
| | | (IMGT) |
| 23 | GLTFERAWMN | CDR-H1 of TJC4 |
| | | (AbM) |
| 24 | RIKRKTDGETTD | CDR-H2 of TJC4 |
| | | (AbM) |
| 25 | SNRAFDI | CDR-H3 of TJC4 |
| | | (AbM) |
| 26 | KSSQSVLYAGNNRNYLA | CDR-L1 of TJC4 |
| | | (AbM) |
| 27 | QASTRAS | CDR-L2 of TJC4 |
| | | (AbM) |
| 28 | QQYYTPPLA | CDR-L3 of TJC4 |
| | | (AbM) |
| 29 | ERAWMN | CDR-H1 of TJC4 |
| | | (Contact) |
| 30 | WVGRIKRKTDGETTD | CDR-H2 of TJC4 |
| | | (Contact) |
| 31 | AGSNRAFD | CDR-H3 of TJC4 |
| | | (Contact) |
| 32 | LYAGNNRNYLAWY | CDR-L1 of TJC4 |
| | | (Contact) |
| 33 | LLINQASTRA | CDR-L2 of TJC4 |
| | | (Contact) |
| 34 | QQYYTPPL | CDR-L3 of TJC4 |
| | | (Contact) |
| 35 | EVQLVESGGGLVKPGGSLRLSCAASGLTFERAWMNWVR | HC of TJC4 |
| | QAPGKGLEWVGRIKRKTDGETTDYAAPVKGRFSISRDDS | |
| | KNTLYLQMNSLKTEDTAVYYCAGSNRAFDIWGQGTMVT | |
| | VSSASTKGPSVFPLAPCSRSTSESTAALGCLVKDYFPEPVT | |
| | VSWNSGALTSGVHTFPAVLQSSGLYSLSSVVTVPSSSLGT | |
| | KTYTCNVDHKPSNTKVDKRVESKYGPPCPPCPAPEFLGG | |
| | PSVFLFPPKPKDTLMISRTPEVTCVVVDVSQEDPEVQFNW | |
| | YVDGVEVHNAKTKPREEQFNSTYRVVSVLTVLHQDWLN | |
| | GKEYKCKVSNKGLPSSIEKTISKAKGQPREPQVYTLPPSQ | |
| | EEMTKNQVSLTCLVKGFYPSDIAVEWESNGQPENNYKTT | |
| | PPVLDSDGSFFLYSRLTVDKSRWQEGNVFSCSVMHEALH | |
| | NHYTQKSLSLSLG | |

1. A method of treating cancer in a subject, comprising administering to the subject an effective amount of an agent that blocks the interaction between CD47 and SIRPα and an effective amount of a KRAS inhibitor, wherein the cancer comprises one or more cancer cells that express a KRAS mutant protein.

2. A method of stimulating phagocytosis of a population of cancer cells by macrophages, comprising contacting the population with an effective amount of an agent that blocks the interaction between CD47 and SIRPα and an effective amount of a KRAS inhibitor, wherein the population of cancer cells comprises one or more cancer cells that express a KRAS mutant protein.

3. The method of claim 1, wherein the agent that blocks the interaction between CD47 and SIRPα is a polypeptide that binds CD47.

4. The method of claim 3, wherein the polypeptide that binds CD47 is anti-CD47 antibody or immunologically active fragment thereof.

5. The method of claim 4, wherein the anti-CD47 antibody or immunologically active fragment thereof is CC-90002, 5F9, LQ001, HLX24, TI-061, AO-176, SRF-231, IBI-188, IMC-002, SHR-1603, STI-6643, ZL-1201, or an immunologically active fragment of any one of the preceding.

6. The method of claim 4, wherein the anti-CD47 antibody or immunologically active fragment thereof comprises three complementarity determining regions (CDRs) of a heavy chain variable domain (V_{H}) set forth in SEQ ID NO: 1 and three CDRs of a light chain variable domain (V_{L}) set forth in SEQ ID NO: 2.

7. The method of claim 4, wherein the anti-CD47 antibody or immunologically active fragment thereof comprises
(a) a V_{H} that comprises (1) a CDR-H1 comprising RAWMN (SEQ ID NO: 5); (2) a CDR-H2 comprising RIKRKTDGETTDYAAPVKG (SEQ ID NO: 6); and (3) a CDR-H3 comprising SNRAFDI (SEQ ID NO: 7) and
(b) a V_{L} that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10), wherein the CDR sequences are defined according to the Kabat numbering system.

8. The method of claim 6, wherein the V_{H} domain of the anti-CD47 antibody or immunologically active fragment thereof comprises an amino acid sequence that has at least 95% identity to SEQ ID NO: 1, and the V_{L} of the anti-CD47 antibody or immunologically active fragment thereof comprises an amino acid sequence that has at least 95% identity to SEQ ID NO: 2.

9. The method of claim 6, wherein the N-terminal amino acid of the V_{H} domain is E and wherein the C-terminal amino acid of the V_{H} domain is S.

10. The method of claim 9, wherein:
(a) the N-terminal amino acid of the V_{H} domain corresponds to position H1 and the C-terminal amino acid of the V_{H} domain corresponds to position H113, according to the Kabat numbering system;
(b) the N-terminal amino acid of the V_{H} domain corresponds to position H1 and the C-terminal amino acid of the V_{H} domain corresponds to position H113, according to the Chothia numbering system; or
(c) the N-terminal amino acid of the V_{H} domain corresponds to position H1 and the C-terminal amino acid of the V_{H} domain corresponds to position H128, according to the IMGT numbering system.

11. The method of claim 6, wherein the N-terminal amino acid of the V_{H} domain corresponds to amino acid 1 of SEQ ID NO: 1, and the C-terminal amino acid of the V_{H} domain corresponds to amino acid 118 of SEQ ID NO: 1.

12. The method of claim 4, wherein the anti-CD47 antibody is a full-length antibody.

13. The method of claim 12, wherein the full length anti-CD47 antibody comprises a human IgG4 Fc region or a variant thereof that comprises an S228P substitution, wherein the amino acid numbering is according to the EU index.

14. The method of claim 12, wherein the full length anti-CD47 antibody comprises a heavy chain that comprises SEQ ID NO: 3 or SEQ ID NO: 35 and a light chain that comprises SEQ ID NO: 4.

15. The method of claim 1, wherein the KRAS inhibitor is a KRAS G12C inhibitor, and wherein the KRAS mutant protein is a KRAS G12C mutant protein.

16. The method of claim 15, wherein the KRAS G12C inhibitor is an antibody, a peptide, a protein, an antisense oligonucleotide, or a small molecule that inhibits the activity of the KRAS G12C mutant protein.

17. The method of claim 16, wherein the KRAS G12C inhibitor is a small molecule.

18. The method of claim 17, wherein the small molecule is selected from the group consisting of: AMG 510, MRTX849, JAB-21822, GDC-6036, JDQ443, D-1553, GH35, GFH925, BPI-421286, and LY3537982.

19. The method of claim 18, wherein the small molecule is AMG 510 or MRTX849.

20. The method of claim 1, wherein the KRAS inhibitor is a KRAS G12D inhibitor, and wherein the KRAS mutant protein is a KRAS G12D mutant protein.

21. The method of claim 20, wherein the KRAS G12D inhibitor is an antibody, a peptide, a protein, an antisense oligonucleotide, or a small molecule that inhibits the activity of the KRAS G12D mutant protein.

22. The method of claim 21, wherein the KRAS G12D inhibitor is a small molecule.

23. The method of claim 22, wherein the small molecule is MTRX1122.

24. The method of claim 1, wherein the cancer is lung cancer, colon cancer, colorectal cancer, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal cancer.

25. The method of claim 1, wherein the cancer is a KRAS G12C cancer.

26. The method of claim 25, wherein the KRAS G12C cancer is lung cancer.

27. The method of claim 26, wherein the lung cancer is lung adenocarcinoma or non-small cell lung cancer (NSCLC).

28. The method of claim 27, wherein the lung cancer is non-small cell lung cancer (NSCLC).

29. The method of claim 1, wherein the cancer is a KRAS G12D cancer.

30. The method of claim 29, wherein the KRAS G12D cancer is colorectal cancer or colon cancer.

31. The method of claim 4, wherein the anti-CD47 antibody or immunologically active fragment thereof and the KRAS inhibitor are administered simultaneously.

32. The method of claim 4, wherein the anti-CD47 antibody or immunologically active fragment thereof and the KRAS inhibitor are administered sequentially.

33. The method of claim 32, wherein the anti-CD47 antibody or immunologically active fragment thereof is administered prior to the KRAS inhibitor.

34. The method of claim 32, wherein the KRAS inhibitor is administered prior to the anti-CD47 antibody or immunologically active fragment thereof.

35. The method of claim 1, wherein the subject is human.

36. The method of claim 2, wherein the one or more cancer cells that express a KRAS mutant protein are lung cancer cells, colon cancer cells, colorectal cancer cells, pancreatic cancer cells, cholangiocarcinoma cells, endometrial cancer cells, ovarian cancer cells, peritoneal cancer cells, bladder cancer cells, gastric cancer cells, thyroid cancer cells, melanoma cells, breast cancer cells, head and neck cancer cells, multiple myeloma cells, acute myeloid leukemia (AML) cells, uterine cancer cells, gastro-esophageal cancer cells, or rectal cancer cells.

37. The method of claim 36, wherein the KRAS inhibitor is a KRAS G12C inhibitor, wherein the one or more cancer cells that express a KRAS mutant protein express a KRAS G12C mutant protein, wherein the one or more cancer cells that express the KRAS G12C mutant protein are lung cancer cells, and wherein the is lung cancer cells are lung adenocarcinoma cells or non-small cell lung cancer (NSCLC) cells.

38. The method of claim 37, wherein the lung cancer cells are NSCLC cells.

39. The method of claim 36, wherein the KRAS inhibitor is a KRAS G12D inhibitor, wherein the one or more cancer cells that express a KRAS mutant protein express a KRAS G12D mutant protein, and wherein the one or more cancer cells that express the KRAS G12D mutant protein are colon cancer cells or colorectal cancer cells.

40. A kit for treating cancer in a human subject comprising:
(a) an agent that blocks the interaction between CD47 and SIRPα, and
(b) instructions for administering an effective amount of the agent and an effective amount of a KRAS inhibitor to a subject who has a cancer that comprises one or more cancer cells that express a KRAS mutant protein.

41. The kit of claim 40, wherein the polypeptide is polypeptide that binds CD47.

42. The kit of claim 41, wherein the polypeptide that binds CD47 is anti-CD47 antibody or immunologically active fragment thereof.

43. The kit of claim 42, wherein the anti-CD47 antibody or immunologically active fragment thereof comprises
(a) a V_{H} that comprises (1) a CDR-H1 comprising RAWMN (SEQ ID NO: 5); (2) a CDR-H2 comprising RIKRKTDGETTDYAAPVKG (SEQ ID NO: 6); and (3) a CDR-H3 comprising SNRAFDI (SEQ ID NO: 7) and
(b) a V_{L} that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10), wherein the CDR sequences are defined according to the Kabat numbering system.

44. The kit of claim 43, wherein the V_{H} domain of the anti-CD47 antibody or immunologically active fragment thereof comprises an amino acid sequence that has at least 95% identity to SEQ ID NO: 1, and the V_{L} of the anti-CD47 antibody or immunologically active fragment thereof comprises an amino acid sequence that has at least 95% identity to SEQ ID NO: 2.

45. The kit of claim 43, wherein the N-terminal amino acid of the V_{H} domain is E and wherein the C-terminal amino acid of the V_{H} domain is S.

46. The kit of claim 42, wherein the anti-CD47 antibody is a full-length antibody.

47. The kit of claim 46, wherein the full length anti-CD47 antibody comprises a heavy chain that comprises SEQ ID NO: 3 or SEQ ID NO: 35 and a light chain that comprises SEQ ID NO: 4.

48. The kit of claim 40, further comprising the KRAS inhibitor.

49. The kit of claim 48, wherein the KRAS inhibitor is a KRAS G12C inhibitor and wherein the cancer comprises one or more cancer cells that express a KRAS G12C mutant protein.

50. The kit of claim 49, wherein the KRAS G12C inhibitor is an antibody, a peptide, a protein, an antisense oligonucleotide, or a small molecule that inhibits the activity of the KRAS G12C mutant protein.

51. The kit of claim 50, wherein the KRAS G12C inhibitor is a small molecule inhibitor.

52. The kit of claim 51, wherein the small molecule is selected from the group consisting of: AMG 510, MRTX849, JAB-21822, GDC-6036, JDQ443, D-1553, GH35, GFH925, BPI-421286, and LY3537982.

53. The kit of claim 52, wherein the small molecule is AMG 510 or MRTX849.

54. The kit of claim 48, wherein the KRAS inhibitor is a KRAS G12D inhibitor and wherein the cancer comprises one or more cancer cells that express a KRAS G12D mutant protein.

55. The kit of claim 54 wherein the KRAS G12D inhibitor is an antibody, a peptide, a protein, an antisense oligonucleotide, or a small molecule that inhibits the activity of the KRAS G12D mutant protein.

56. The kit of claim 55, wherein the KRAS G12D inhibitor is a small molecule inhibitor.

57. The kit of claim 56, wherein the small molecule MRTX1133.

58. The kit of claim 40, wherein the cancer that comprises one or more cancer cells that express a KRAS mutant protein is lung cancer, colon adenocarcinoma, colorectal adenocarcinoma, pancreatic cancer, cholangiocarcinoma, endometrial cancer, ovarian cancer, peritoneal cancer, bladder cancer, gastric cancer, thyroid cancer, melanoma, breast cancer, head and neck cancer, multiple myeloma, acute myeloid leukemia (AML), uterine cancer, gastro-esophageal cancer, or rectal adenocarcinoma.

59. A pharmaceutical composition comprising an agent that blocks the interaction between CD47 and SIRPα and a KRAS inhibitor.

60. The pharmaceutical composition of claim 59, wherein the agent that blocks the interaction between CD47 and SIRPα is an anti-CD47 antibody or immunologically active fragment thereof.

61. The pharmaceutical composition of claim 60, wherein the anti-CD47 antibody or immunologically active fragment thereof is CC-90002, 5F9, LQ001, HLX24, TI-061, AO-176, SRF-231, IBI-188, IMC-002, SHR-1603, STI-6643, ZL-1201, or an immunologically active fragment.

62. The pharmaceutical composition of claim 59, wherein the anti-CD47 antibody or immunologically active fragment thereof comprises
(a) a V_{H} that comprises (1) a CDR-H1 comprising RAWMN (SEQ ID NO: 5); (2) a CDR-H2 comprising RIKRKTDGETTDYAAPVKG (SEQ ID NO: 6); and (3) a CDR-H3 comprising SNRAFDI (SEQ ID NO: 7) and
(b) a V_{L} that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10), wherein the CDR sequences are defined according to the Kabat numbering system.

63. The pharmaceutical composition of claim 59, wherein the KRAS inhibitor is a KRAS G12C inhibitor.

64. The pharmaceutical composition of claim 63, wherein the KRAS G12C inhibitor is selected from the group consisting of AMG 510, MRTX849, JAB-21822, GDC-6036, JDQ443, D-1553, GH35, GFH925, BPI-421286, and LY3537982.

65. The pharmaceutical composition of claim 59, wherein the KRAS inhibitor is a KRAS G12D inhibitor.

66. The pharmaceutical composition of claim 65, wherein the KRAS G12D inhibitor is MTRX1133.
